(19) United States
(12) Patent Application Publication
(10) Pub. No.: US 2024/0362775 A1
(43) Pub. Date: Oct. 31, 2024

(54) SYSTEMS, METHODS, AND APPARATUSES FOR IMPLEMENTING IMPROVED GENERALIZABILITY, TRANSFERABILITY, AND ROBUSTNESS THROUGH MODALITY UNIFICATION, FUNCTION INTEGRATION, AND ANNOTATION AGGREGATION

(71) Applicant: Arizona Board of Regents on behalf of Arizona State University, Scottsdale, AZ (US)

(72) Inventor: Jianming Liang, Scottsdale, AZ (US)

(21) Appl. No.: 18/641,225

(22) Filed: Apr. 19, 2024

Related U.S. Application Data
(60) Provisional application No. 63/498,673, filed on Apr. 27, 2023.

(51) Int. Cl. G06T 7/00; G06T 7/12; G06V 10/764; G16H 30/20; G16H 30/40; G16H 50/20

(52) U.S. Cl. CPC G06T 7/0012; G06T 7/12; G06V 10/764; G16H 30/20; G16H 30/40; G16H 50/20; G06T 2207/30096

(57) ABSTRACT

Medical image data is received at the system from a plurality of public or private datasets; An AI model is trained on the datasets to learn image classification and outputs (i) an image-level classification function, (ii) an object-level classification function, and (iii) a plurality of image classification weights; the AI model is trained on the datasets to learn image localization and output an object localization function and image localization weights; the AI model is trained on the datasets to learn image segmentation and output an object segmentation function and image segmentation weights; each of the image classification weights is integrated with the image localization weights and the image segmentation weights into a single pre-trained AI model; each of the image-level classification function, the object-level classification function, the object localization function and the object segmentation function are integrated into a single pre-trained AI model for use with medical image analysis.

### CLAIM OF PRIORITY

This application claims the benefit of U.S. Provisional Patent Application No. 63/498,673, filed Apr. 27, 2023, entitled “SYSTEMS, METHODS, AND APPARATUSES FOR IMPLEMENTING IMPROVED GENERALIZABILITY, TRANSFERABILITY, AND ROBUSTNESS THROUGH MODALITY UNIFICATION, FUNCTION INTEGRATION, AND ANNOTATION AGGREGATION”, the disclosure of which is incorporated by reference herein in its entirety.

### GOVERNMENT RIGHTS AND GOVERNMENT AGENCY SUPPORT NOTICE

This invention was made with government support under R01 HL128785 awarded by the National Institutes of Health. The government has certain rights in the invention.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### TECHNICAL FIELD

Embodiments of the invention relate generally to the field of medical imaging and analysis using convolutional neural networks and transformers for the classification and annotation of medical images, and more particularly, to systems, methods, and apparatuses for implementing improved generalizability, transferability, and robustness through modality unification, function integration, and annotation aggregation, in the context of medical image analysis.

### BACKGROUND

The subject matter discussed in the background section should not be assumed to be prior art merely because it is mentioned in the background section. Similarly, a problem mentioned in the background section or associated with the subject matter of the background section should not be assumed to have been previously recognized in the prior art. The subject matter in the background section merely represents different approaches, which in and of themselves may also correspond to embodiments of the claimed inventions.

Machine learning models have various applications to automatically process inputs and produce outputs considering situational factors and learned information to improve output quality. One area where machine learning models, and neural networks in particular, provide high utility is in the field of processing medical images.

Within the context of machine learning and deep learning specifically, a Convolutional Neural Network (CNN, or ConvNet) is a class of deep neural networks very often applied to analyzing visual imagery. Convolutional Neural Networks are regularized versions of multilayer perceptrons. Multilayer perceptrons are fully connected networks, such that each neuron in one layer is connected to all neurons in the next layer, a characteristic which often leads to a problem of overfitting of the data and the need for model regularization. Convolutional Neural Networks also seek to apply model regularization, but with a distinct approach. Specifically, CNNs take advantage of the hierarchical pattern in data and assemble more complex patterns using smaller and simpler patterns. Consequently, on the scale of connectedness and complexity, CNNs are on the lower extreme.

Unfortunately, prior known techniques involving unsupervised or supervised learning modes fail to provide integrated artificial intelligence (AI) models capable of providing acceptable results.

What is needed is an improved technique for model integration capable of producing superior results across classification, localization, segmentation modalities, and different target tasks.

The present state of the art may therefore benefit from the systems, methods, and apparatuses for implementing improved generalizability, transferability, and robustness through modality unification, function integration, and annotation aggregation, in the context of medical image analysis, as is described herein.

### BRIEF DESCRIPTION OF THE DRAWINGS

Embodiments are illustrated by way of example, and not by way of limitation, and can be more fully understood with reference to the following detailed description when considered in connection with the figures in which:

FIGS. 1A, 1B, and 1C depict various exemplary functions for the major image analysis tasks.

FIGS. 2A, 2B, 2C, and 2D depict exemplary functions for the major image analysis tasks.

FIGS. 3A, 3B, 3C, 3D, 3E, 3F, and 3G depict exemplary functions for the major image analysis tasks.

FIGS. 4A, 4B, and 4C depict simple example models utilizing warm-up exercises including functions to classify animals between cats and dogs with explanations why deep learning can perform such tasks.

FIGS. 5A, 5B, 5C, 5D, and 5E depict simple example models utilizing warm-up exercises including functions to classify and to estimate the age of a human.

FIGS. 6A and 6B depict simple example models utilizing warm-up exercises including functions to classify and to estimate the human weights, heights, and races.

FIGS. 7A, 7B, and 7C depict simple example supervised learning models utilizing warm-up exercises including functions for segmentation and localization.

FIGS. 8A, 8B, 8C, 8D, 8E, 8F, 8G, 8H, 8I, 8J, 8K, and 8L depict example un-supervised learning models utilizing warm-up exercises.

FIGS. 9A, and 9B depict example un-supervised learning models utilizing warm-up exercises.

FIGS. 10A, 10B, 10C, 10D, 10E, 10F, 10G 10H, 10I, 10J, 10K, 10L, 10M, 10N, 10O, 10P, 10Q, 10R, 10S, and 10T depict various model integration objectives.

### DETAILED DESCRIPTION

Described herein are systems, methods, and apparatuses for implementing improved generalizability, transferability, and robustness through modality unification, function integration, and annotation aggregation, in the context of medical image analysis.

### INTRODUCTION

Described herein are means for an all-in-one approach to achieving superior generalizability, transferability, and robustness through the implementation of modality unification, function integration, and annotation aggregation.

Deep learning offers expert-level and sometimes even super-expert-level performance, yet, achieving such performance demands a massively annotated dataset for training.

In the context of Medical Imaging, there are a variety of modalities and many applications, yielding numerous (different) datasets. Unfortunately, these datasets are individually small, inconsistent in disease coverage, and heterogeneous in expert annotations.

Therefore, described herein is a novel method that can utilize many different image datasets and their associated heterogeneous annotations for classification, localization, and segmentation across imaging modalities to pre-train generic source models that are more robust, generalizable, and transferable to application-specific target tasks. Stated differently, the methodologies set forth herein provide a novel method that pre-train deep models by utilizing all accessible annotations for classification, localization, and segmentation across imaging modalities, so that the pre-trained models are more generalizable and transferred to a variety of image analysis tasks for offering superior and robust performance.

From a high level, the disclosed techniques (1) Integrate Functions, providing: Image-level classification, Organ/Lesion localization, Lesion-level classification, Organ/Lesion segmentation; (2) Unify Modalities, including: X-rays, CT (computed tomography), MRI, Colonoscopy, etc.; and (3) Aggregate Annotations, including: Image-level label, Organ/Lesion bounding box, Organ/lesion markers, Organ/Lesion masks.

Benefits from Modality Unification, Function Integration, and Annotation Aggregation include at least: enlarged data size, diversified patient populations, accrued knowledge from more experts, trained generic source models which are strong in generalizability, transferability, and robustness, yield application-specific target models which are superior in task performance and robust in imbalanced datasets (biases).

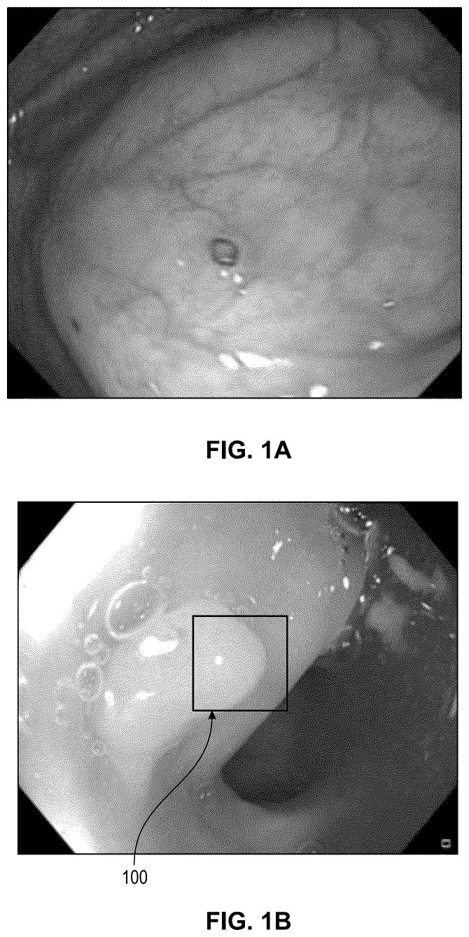

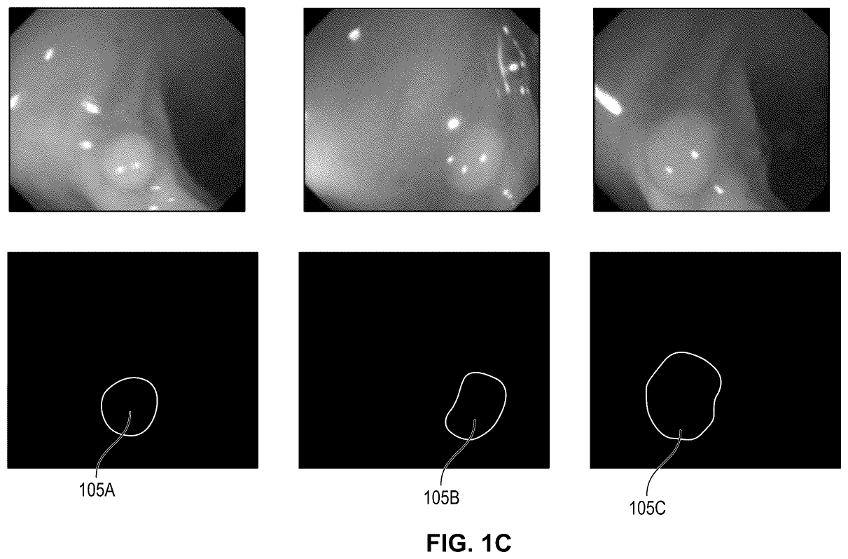

FIG. 1A depicts exemplary functions for the major image analysis tasks including monitoring monitor video quality. Specifically shown here is the capability for automatically monitoring the colonoscopic video quality.

FIG. 1B depicts exemplary functions for the major image analysis tasks including the ability to localize polyps such as the polyp identified in box 100. Specifically shown here is the capability for automatically alerting the physician to possible polyps.

FIG. 1C depicts exemplary functions for the major image analysis tasks including the ability to segment polyps. Specifically shown here is the capability for automatically estimating the size of various polyps as indicated by the irregular circles of different dimensions at 105A, 105B, and 105C that estimate the size of the respective polyps in the corresponding pictures positioned directly above.

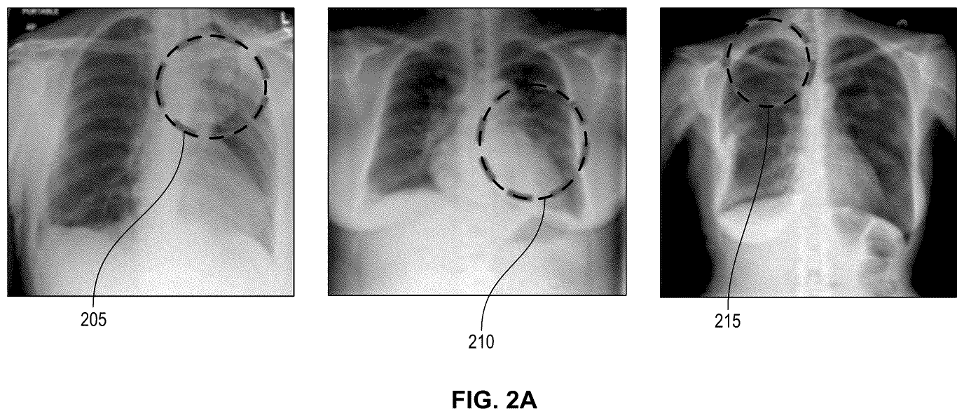

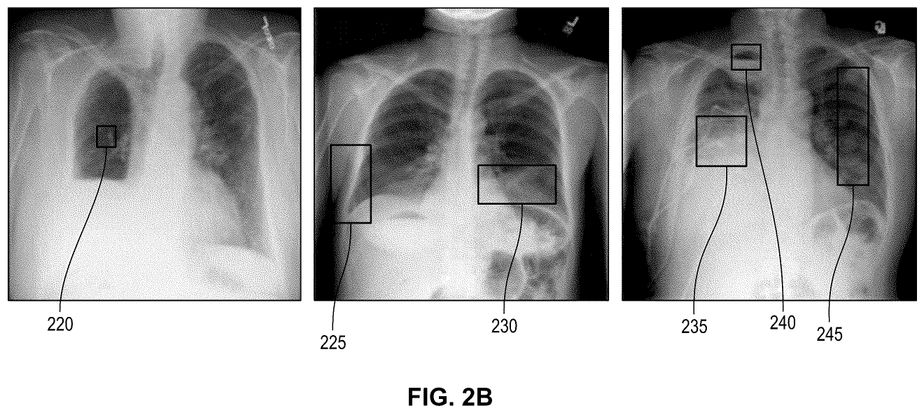

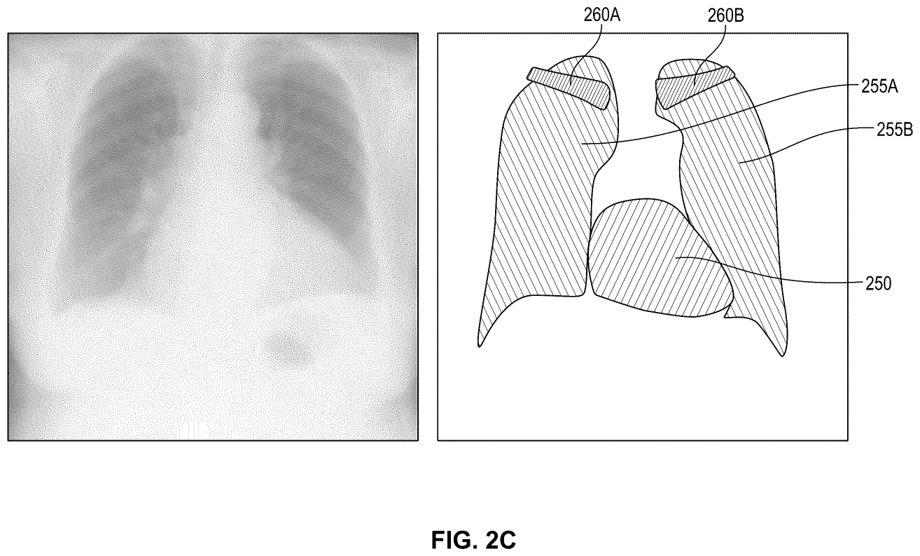

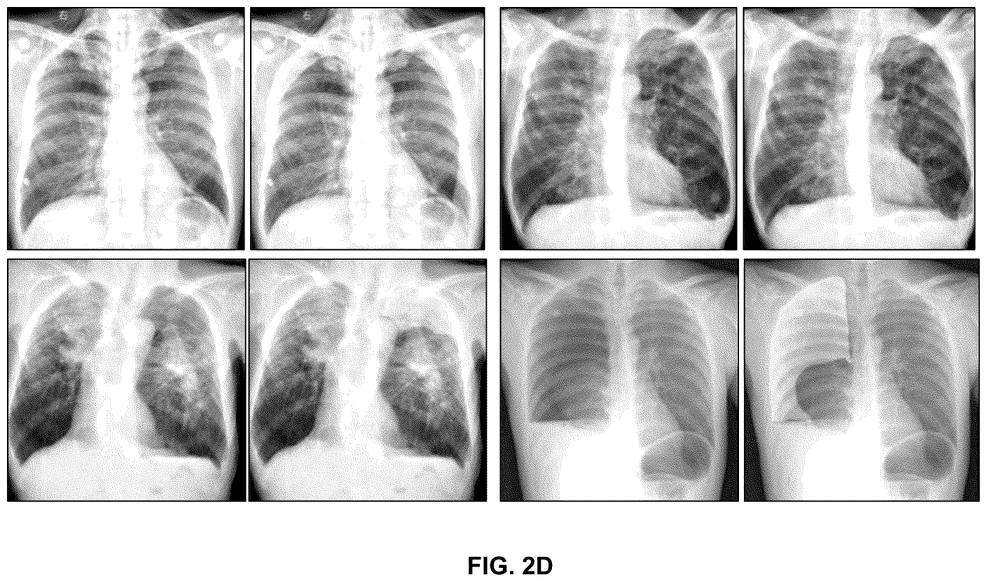

FIG. 2A depicts exemplary functions for the major image analysis tasks including analyzing chest images. Specifically shown here is the capability for automatically classifying lung diseases, including Atelectasis at 205, Pneumonia at 210, and Pneumothorax at 215.

FIG. 2B depicts exemplary functions for the major image analysis tasks including analyzing chest images. Specifically shown here is the capability for automatically localizing lung diseases such as a nodule 220, effusion 225, infiltrate 230, infiltrate 235, pneumothorax 240, and mass 245 in chest X-rays.

FIG. 2C depicts exemplary functions for the major image analysis tasks including analyzing chest images. Specifically shown here is the capability for automatically segmenting the heart 250, lungs 255A, 255B, and clavicle bones 260A, 260B.

FIG. 2D depicts exemplary functions for the major image analysis tasks including analyzing chest images. Specifically shown here is the capability for automatically segmenting pneumothorax.

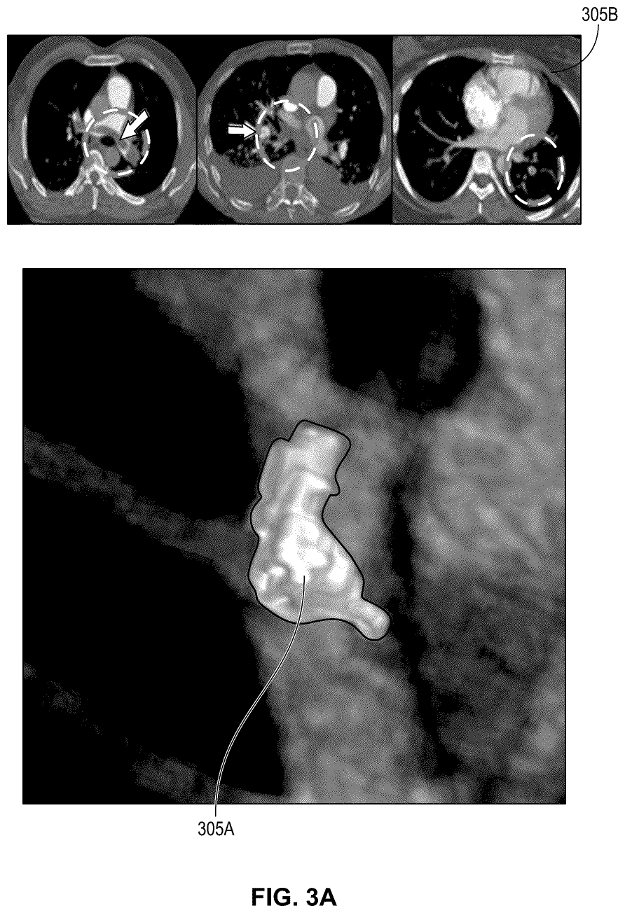

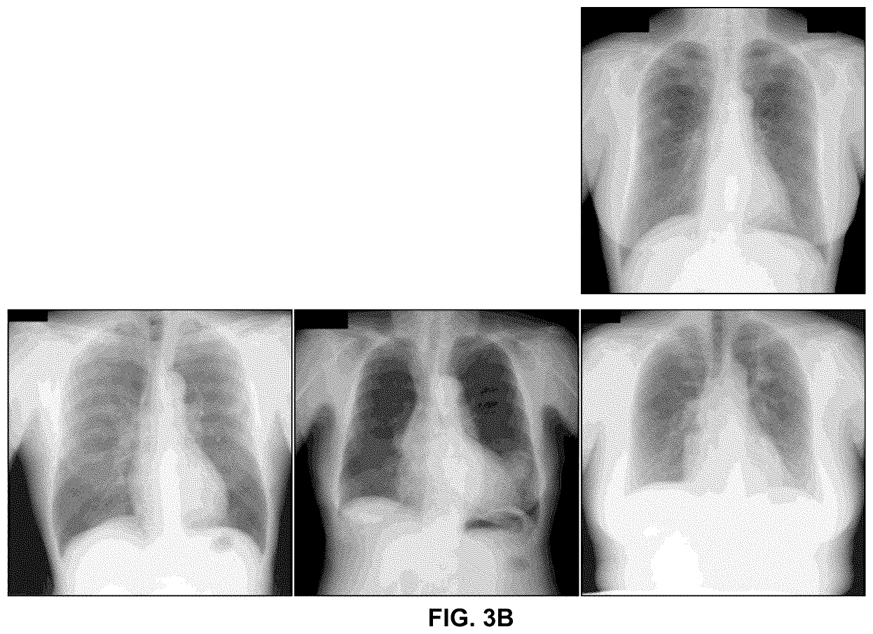

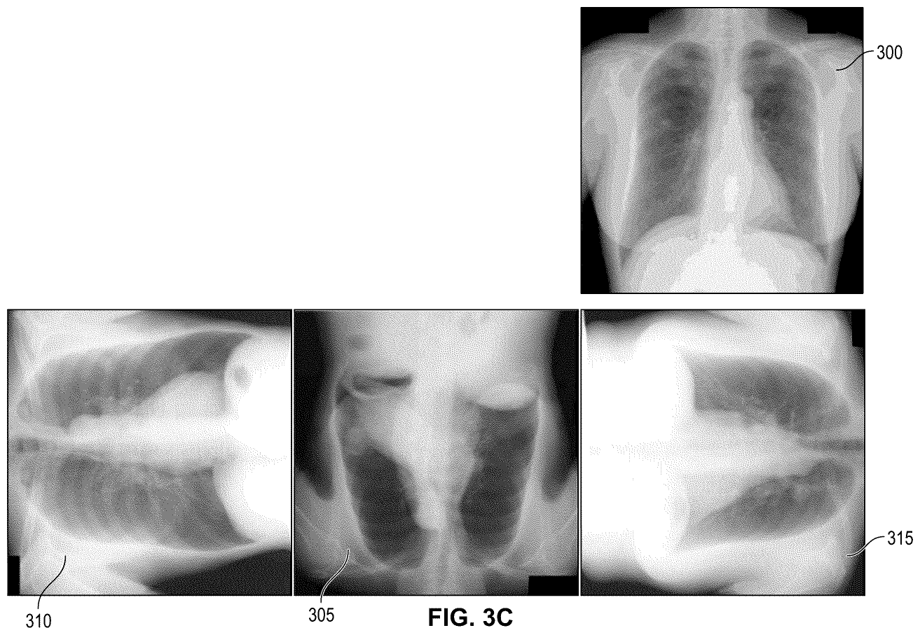

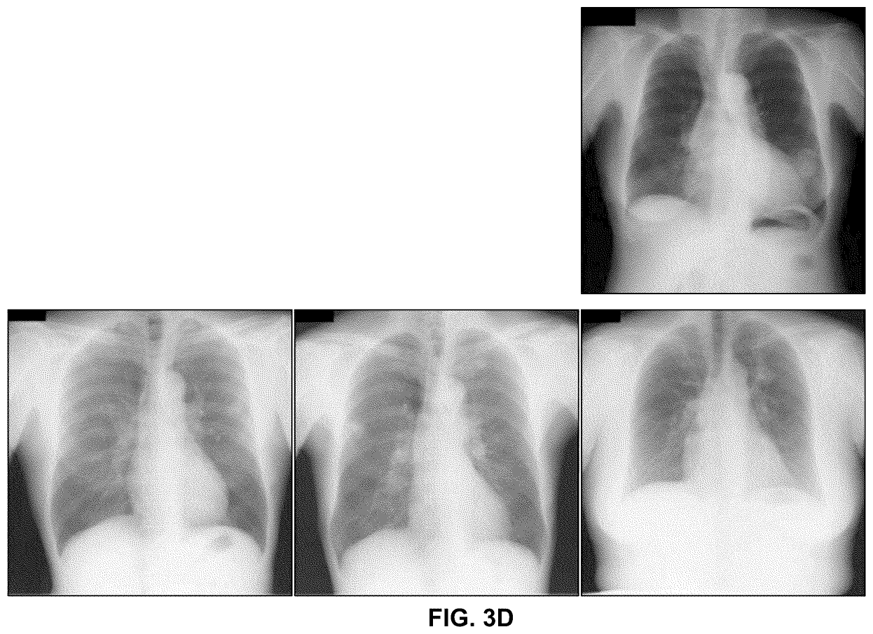

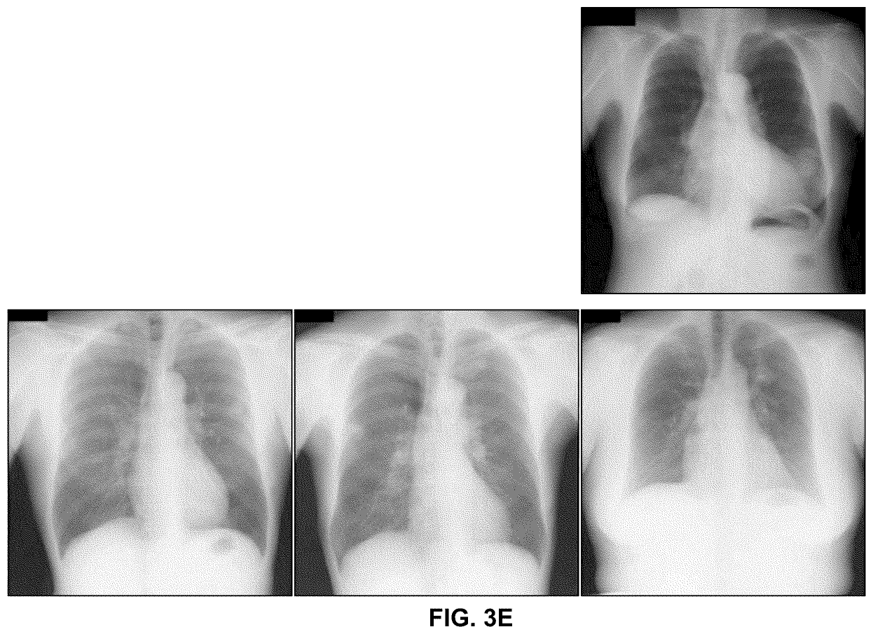

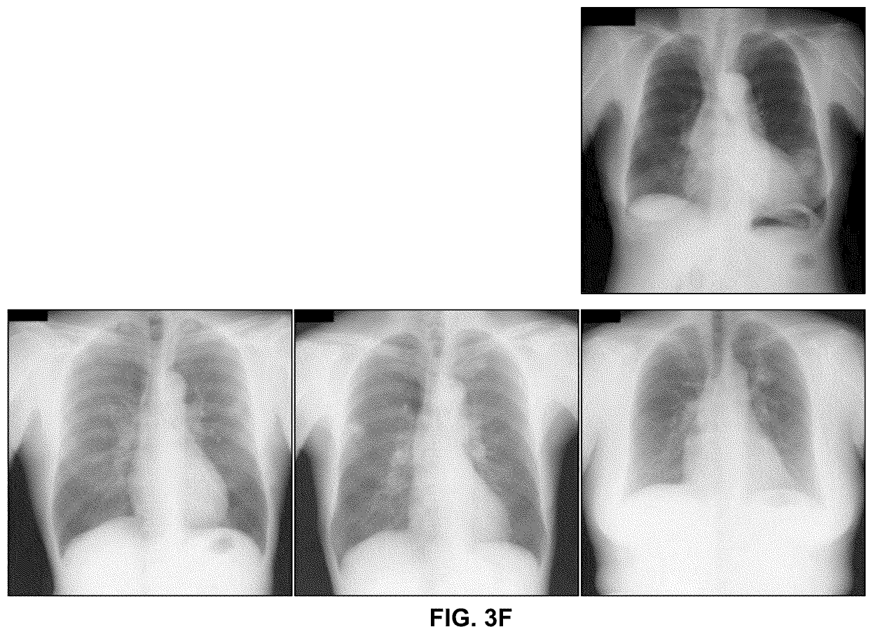

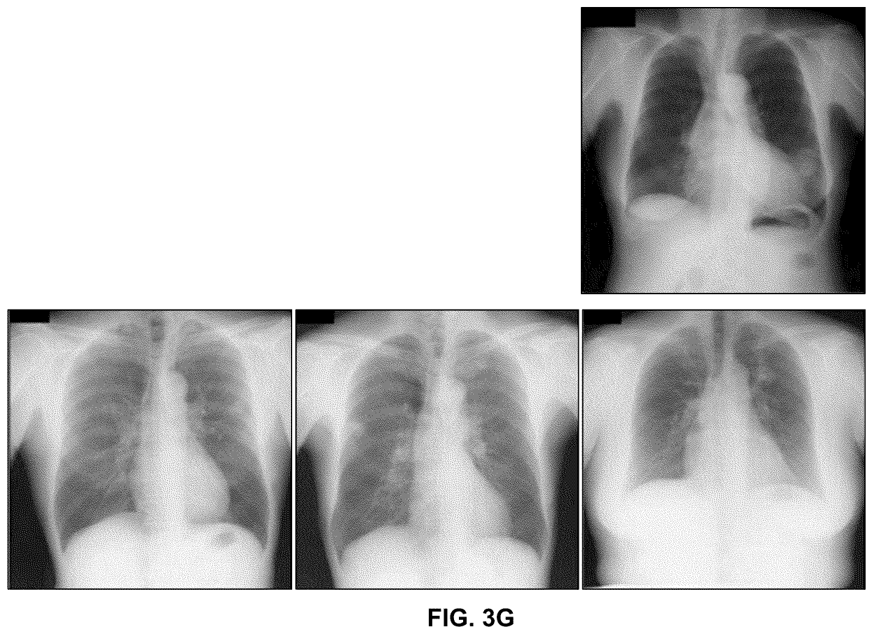

FIG. 3A depicts exemplary functions for the major image analysis tasks including analyzing chest CT. Specifically shown here is the capability for automatically localizing and segmenting pulmonary embolism (PE) 305A, including pulmonary embolism detection and segmentation and pulmonary embolism segmentation and visualization 305B.

As shown here, function examples include major image analysis tasks, image classification object (organ/lesion) localization, and object (organ/lesion) segmentation.

Function examples for each are as follows: major image analysis tasks include image classification, such as colonoscopy video quality assessment (colonoscopy), polyp frame classification (colonoscopy), lung disease classification (chest X-rays and chest CT). Object (Organ/Lesion) localization include: polyp localization (colonoscopy) and lung disease localization (VinDr CXR). Object (Organ/Lesion) segmentation includes: polyp segmentation (colonoscopy) and lung/heart/clavicle bone segmentation (chest X-rays).

FIG. 3B depicts simple example models utilizing ResNet18 or Swin Tiny model variants. Specifically shown are simple examples including warm-up exercises to read and write chest X-rays, using Practice_PNG and JPG.zip and Practice_DICOM.zip, and PNG, JPG, and DICOM.

FIG. 3C depicts simple example models utilizing warm-up exercises including functions to classify orientations via Directions01.zip as up (300), down (305), left (310), and right (315).

FIG. 3D depicts simple example models utilizing warm-up exercises including functions to classify human gender as either male or female.

FIG. 3E depicts simple example models utilizing warm-up exercises including functions to classify human gender as either male or female specifically via deep learning.

FIG. 3F depicts simple example models utilizing warm-up exercises including functions to classify human gender as either male or female via functions Gender01.zip, resulting in classification as either male or female.

FIG. 3G depicts simple example models utilizing warm-up exercises including functions to classify human gender with AI-based explanations on why deep learning can perform such tasks, with reference to advanced AI explainability for PyTorch (e.g., github.com/jacobgil/pytorch-grad-cam).

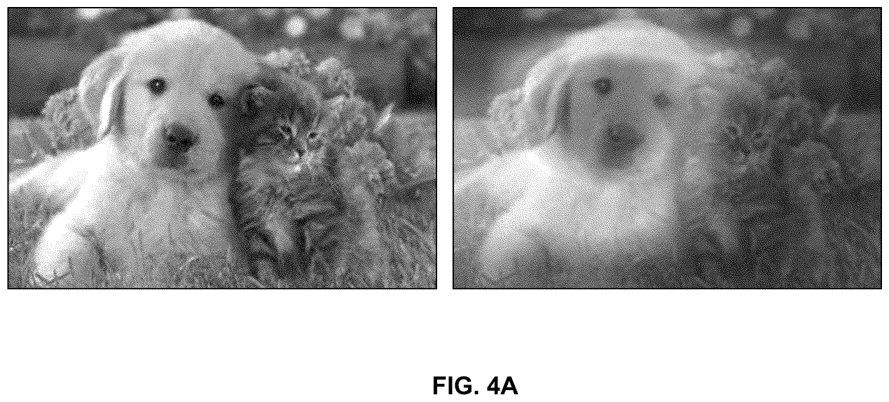

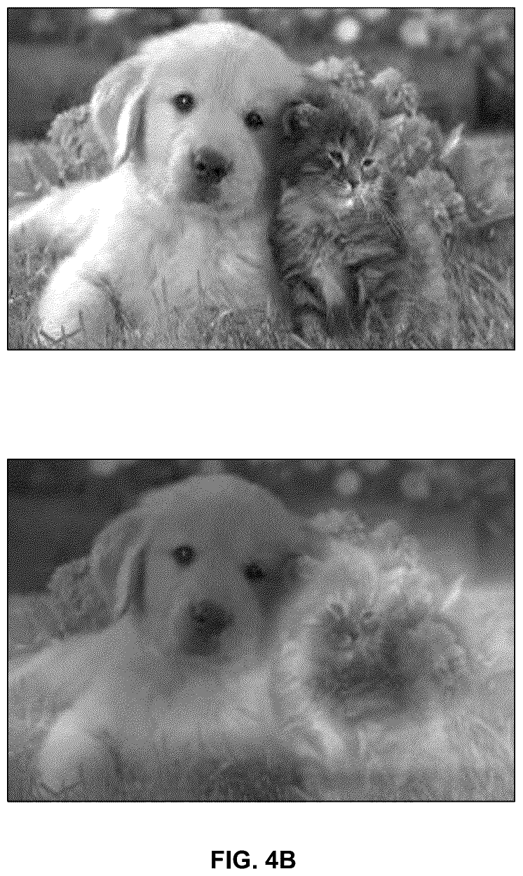

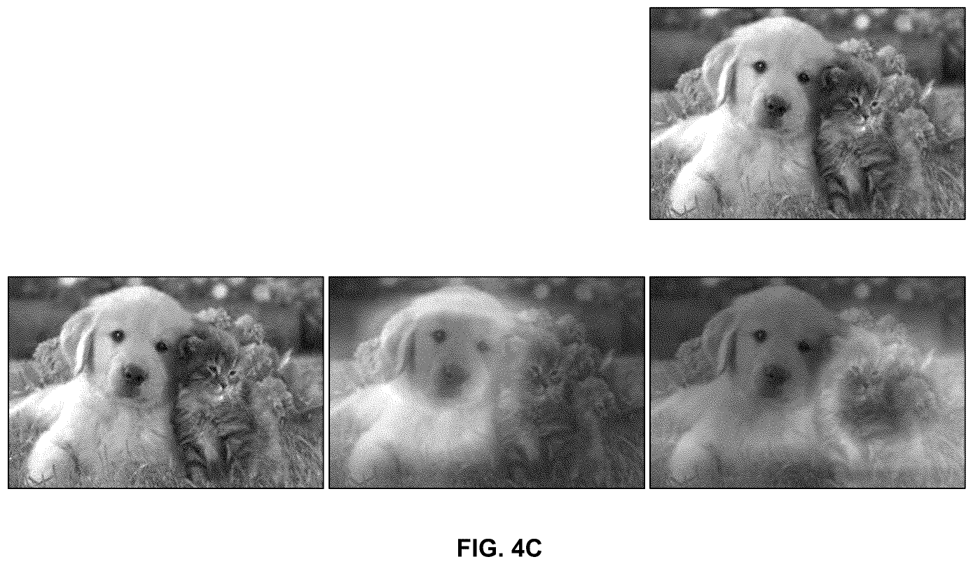

FIGS. 4A, 4B, and 4C depict simple example models utilizing warm-up exercises including functions to classify animals between cats and dogs with explanations why deep learning can perform such tasks.

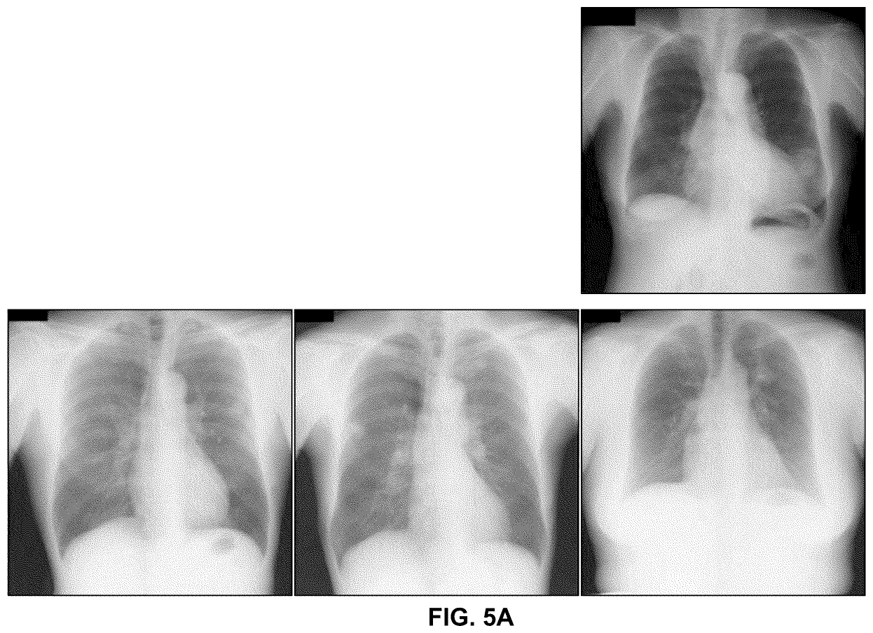

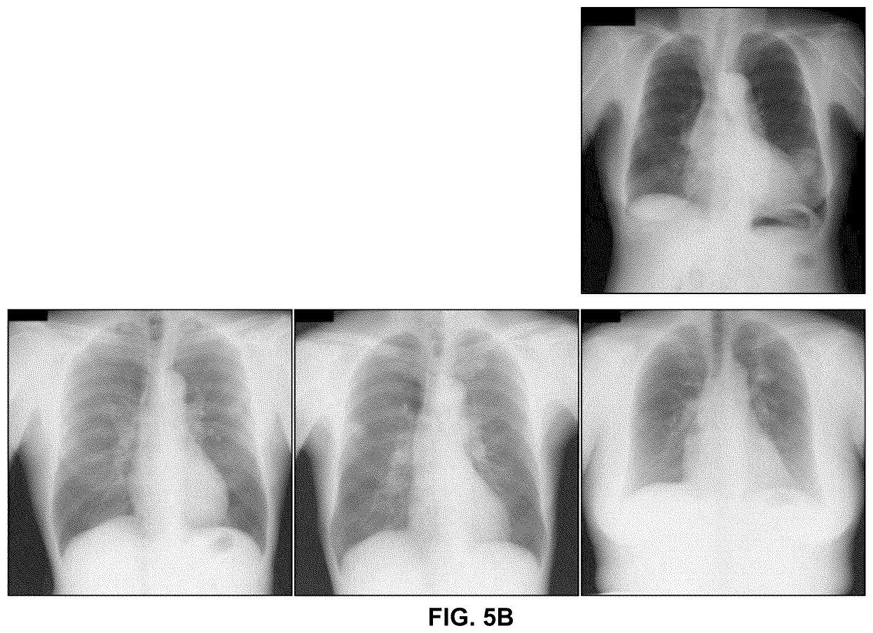

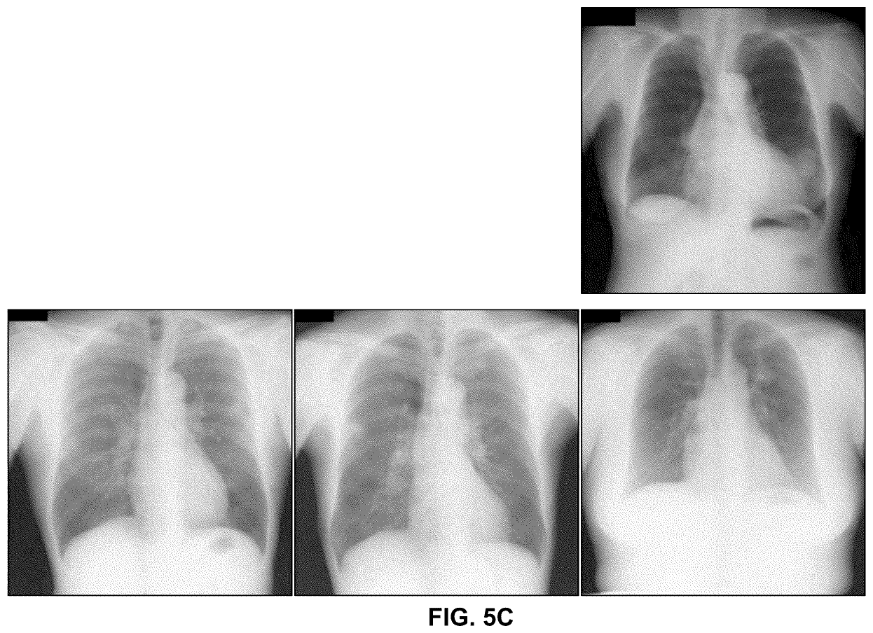

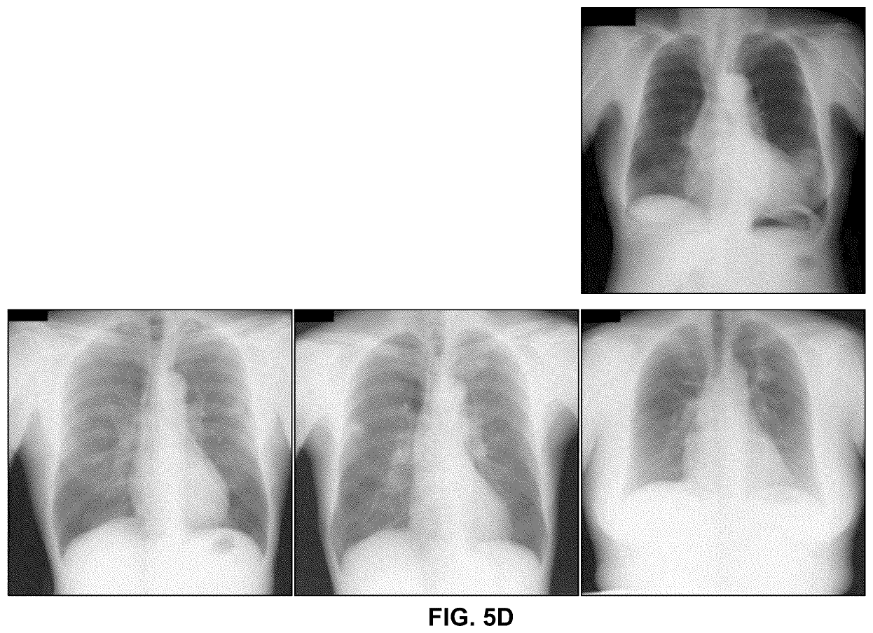

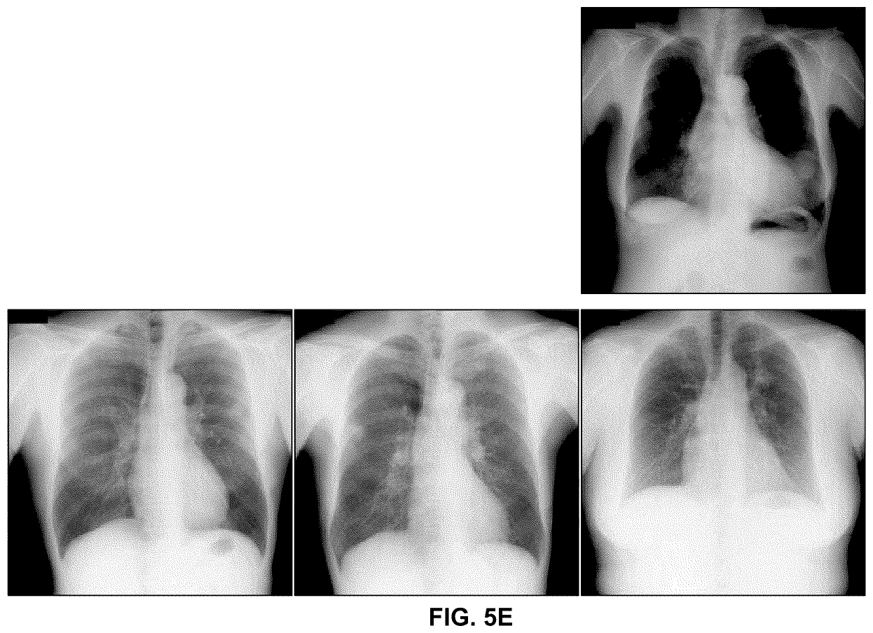

FIGS. 5A, 5B, 5C, 5D, and 5E depict simple example models utilizing warm-up exercises including functions to classify and estimate the age of a human. Specifically shown are estimating tricks executable via CAM and XPAge01_RGB.zip functions to output an age estimate between 16˜89.

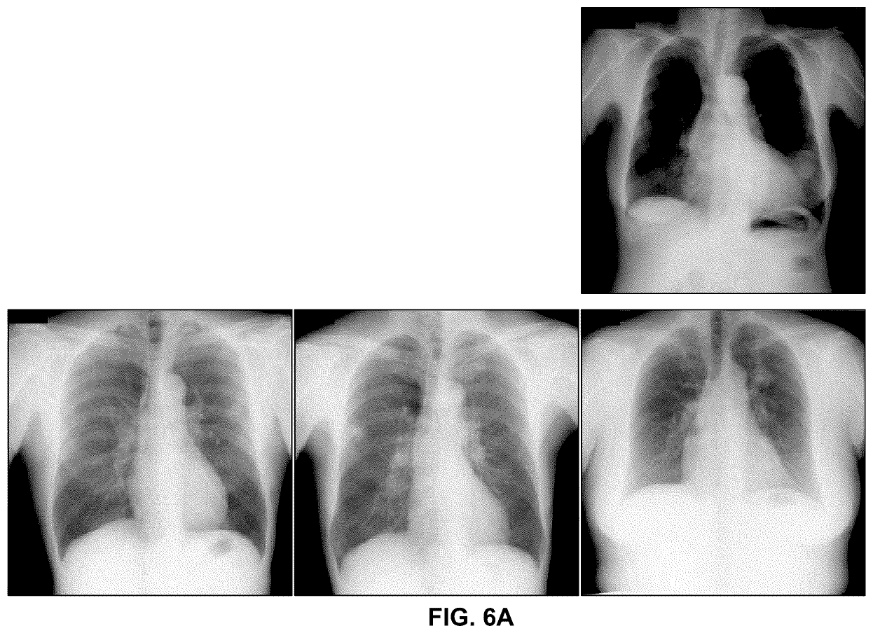

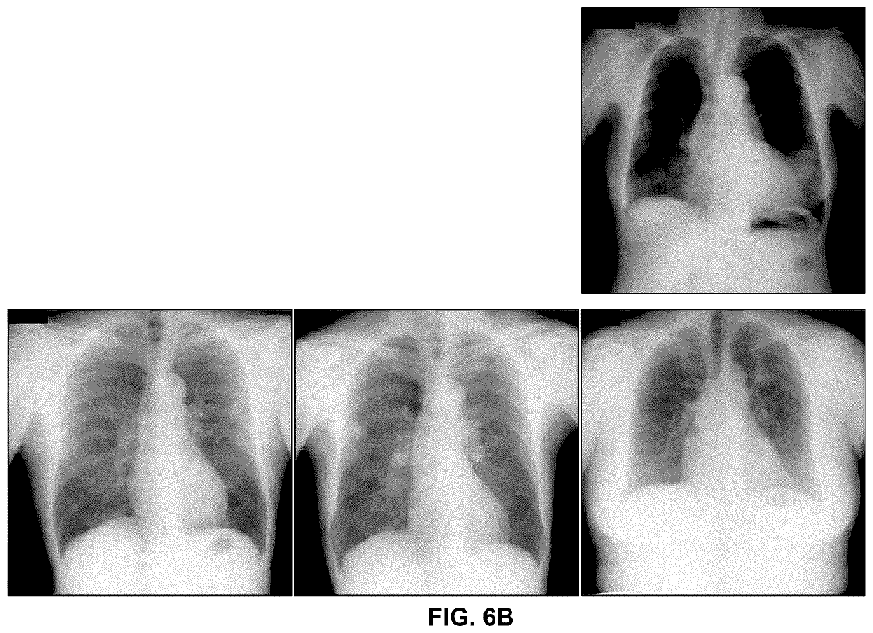

FIGS. 6A and 6B depict simple example models utilizing warm-up exercises including functions to classify and to estimate the human weights, heights, and races. Specifically shown are estimating tricks executable via a MIMIC dataset to estimate human weights, heights, and races.

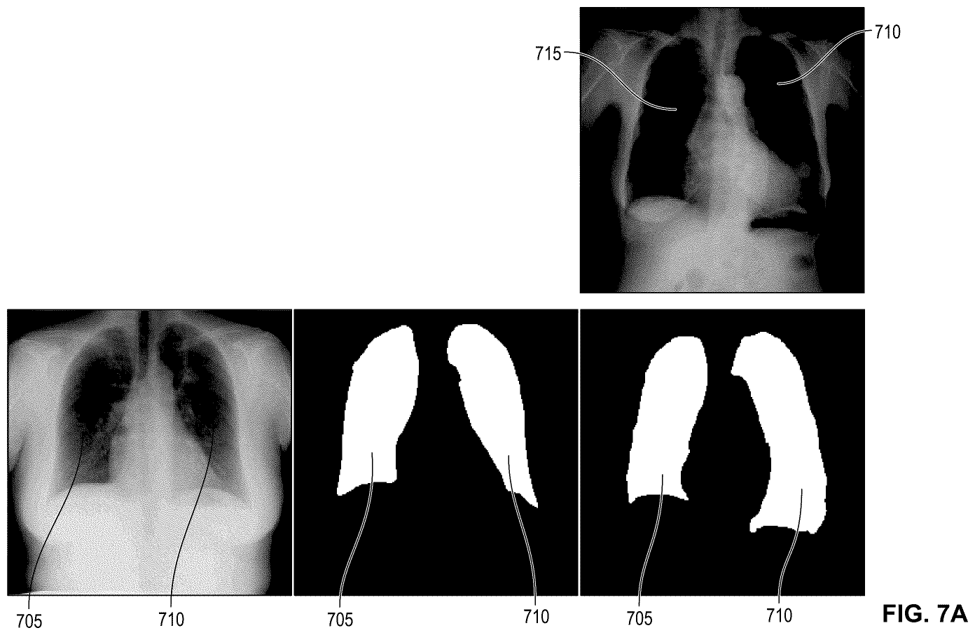

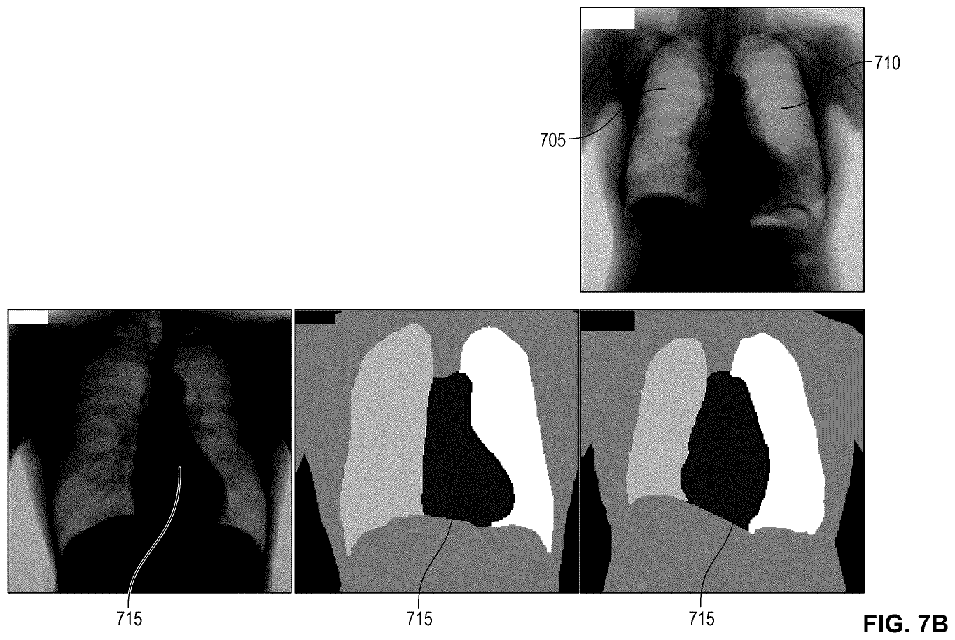

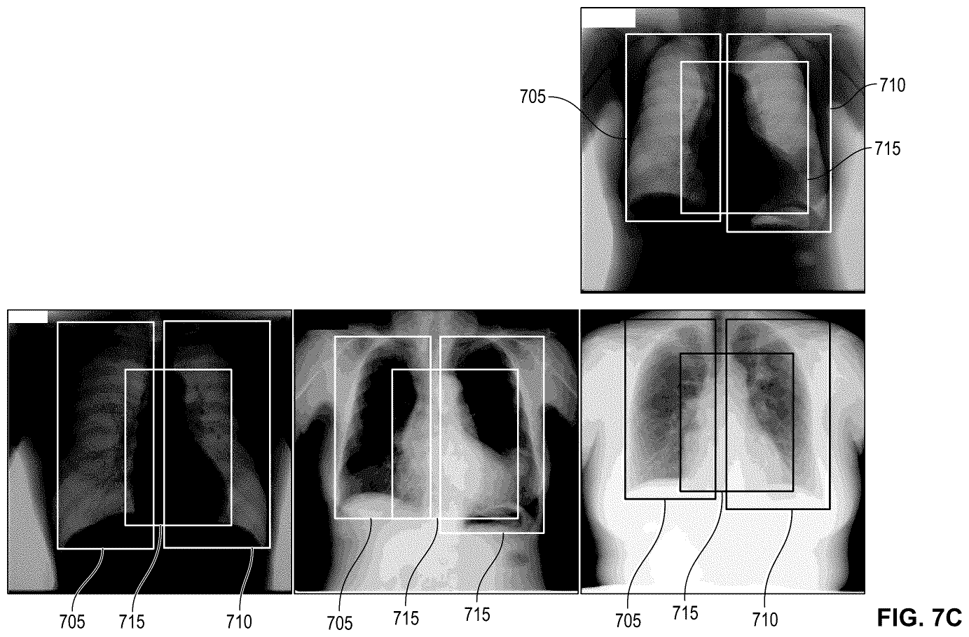

FIGS. 7A, 7B, and 7C depict simple exemplary supervised learning models utilizing warm-up exercises including functions for segmentation and localization. Specifically shown are techniques for semantic lung segmentation via Segmentation01.zip to determine right lungs 705 and left lungs 710: same label (255), and instance organ segmentation created from Segmentation02.zip identifying the heart 715, right lung 705, left lung 710, chest area, and the outside of the body, and techniques to localize organs (lesions) created from Segmentation02.zip to identify heart 715, right lung 705, and left lung 710.

More particularly, the warm-up exercises include supervised learning functions to classify orientations as up, down, left, and right; functions to classify genders (races) as male and female; functions to estimate ages (heights/weights) as being between 16˜89; functions for semantic lung segmentation, to determine lung masks; functions for instance organ segmentation to identify organ masks; and functions to localize organs to establish organ bounding boxes.

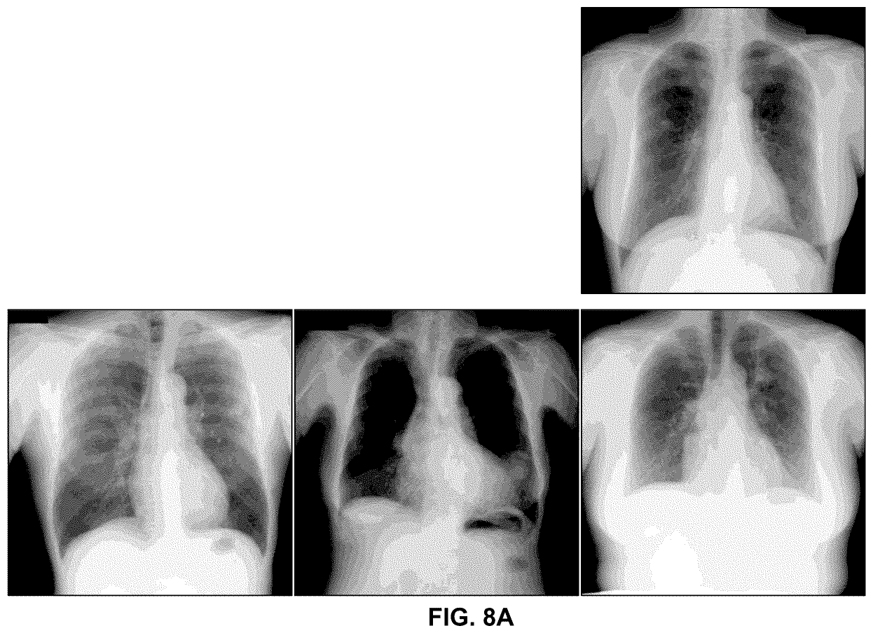

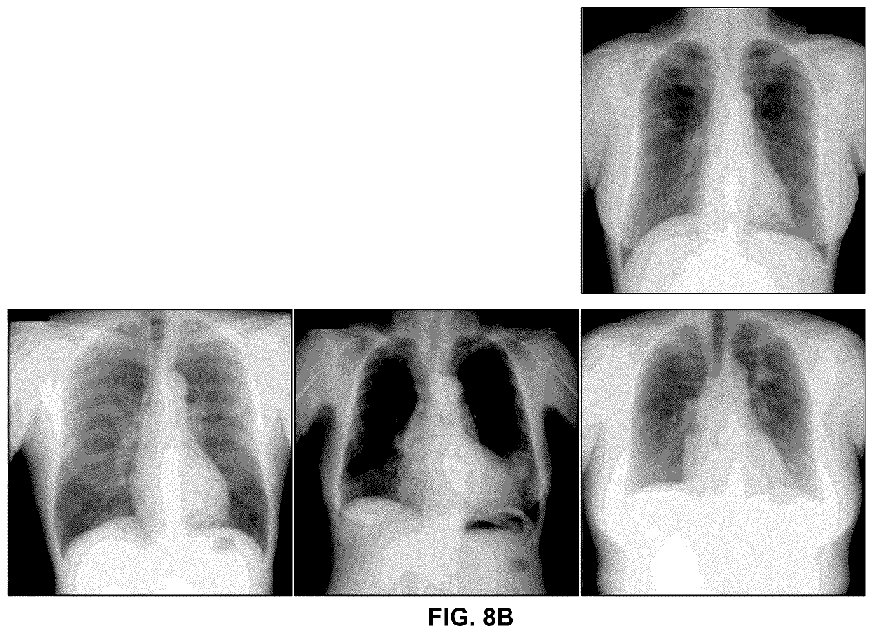

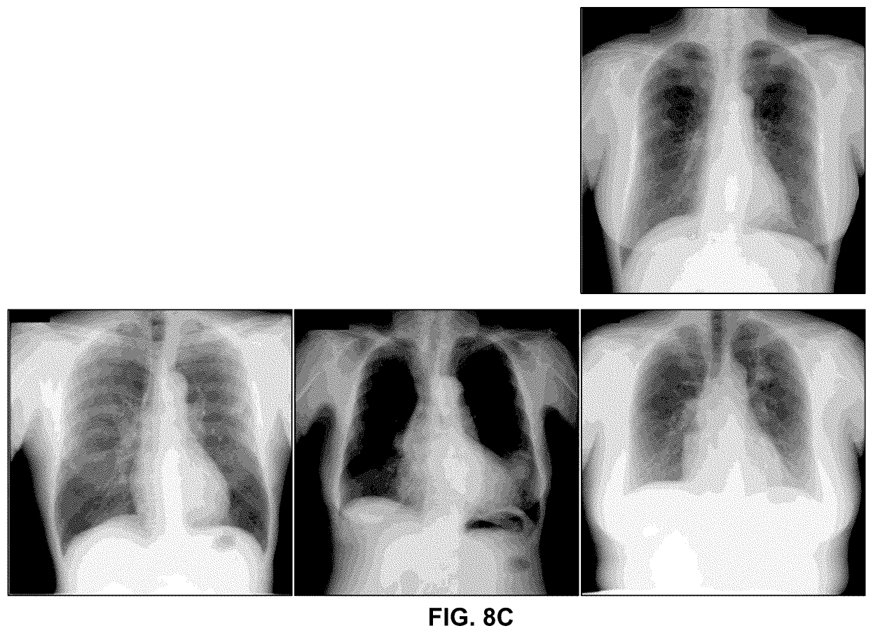

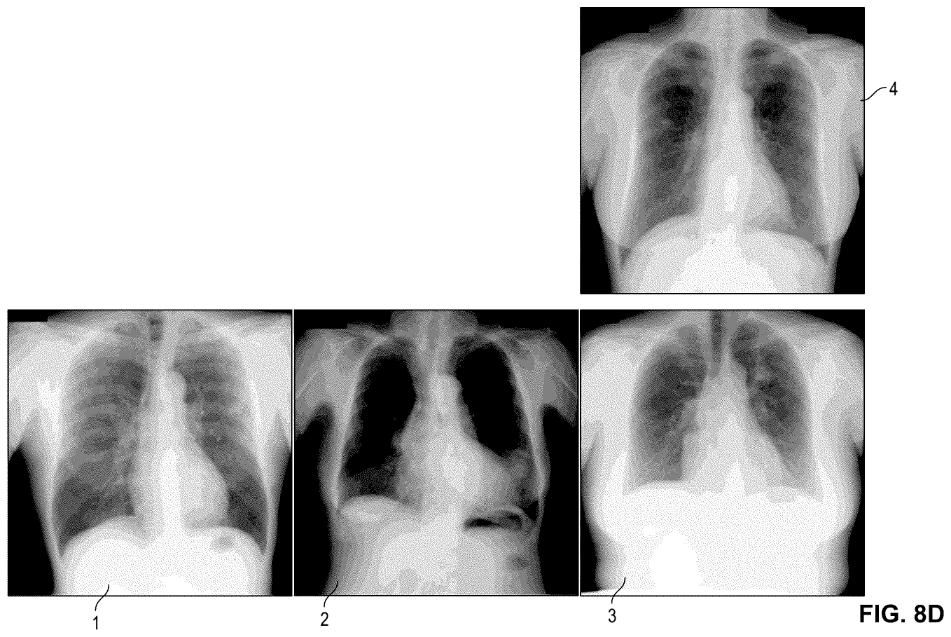

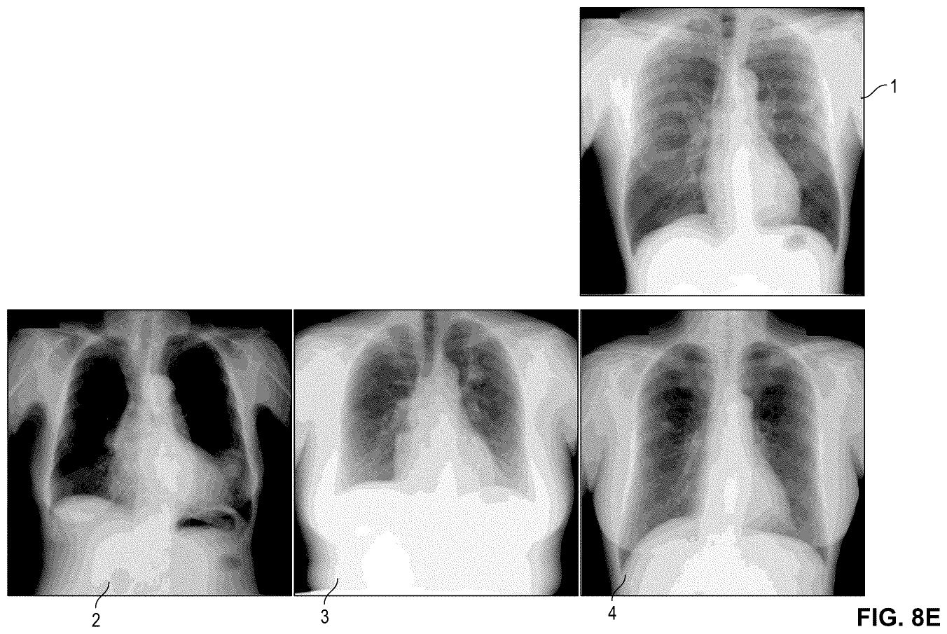

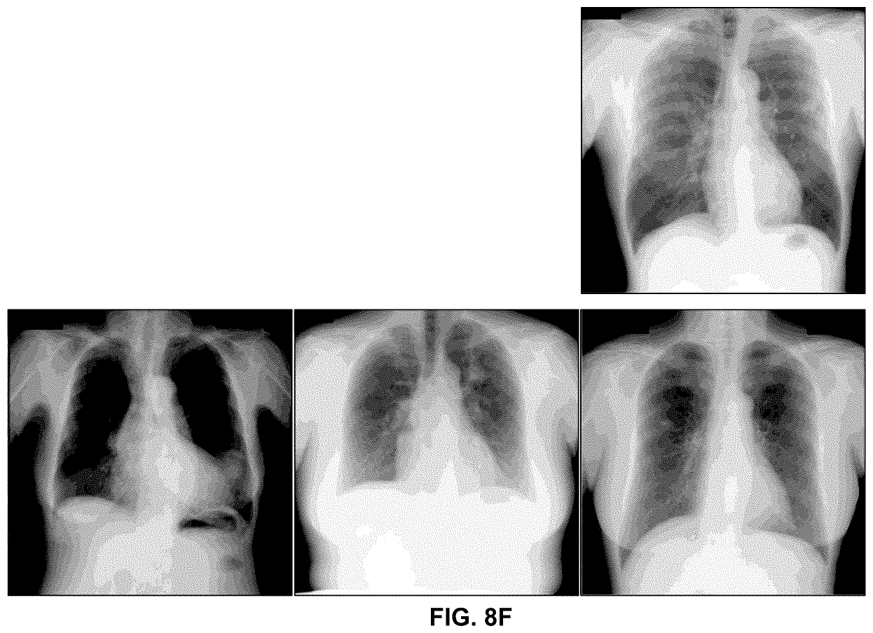

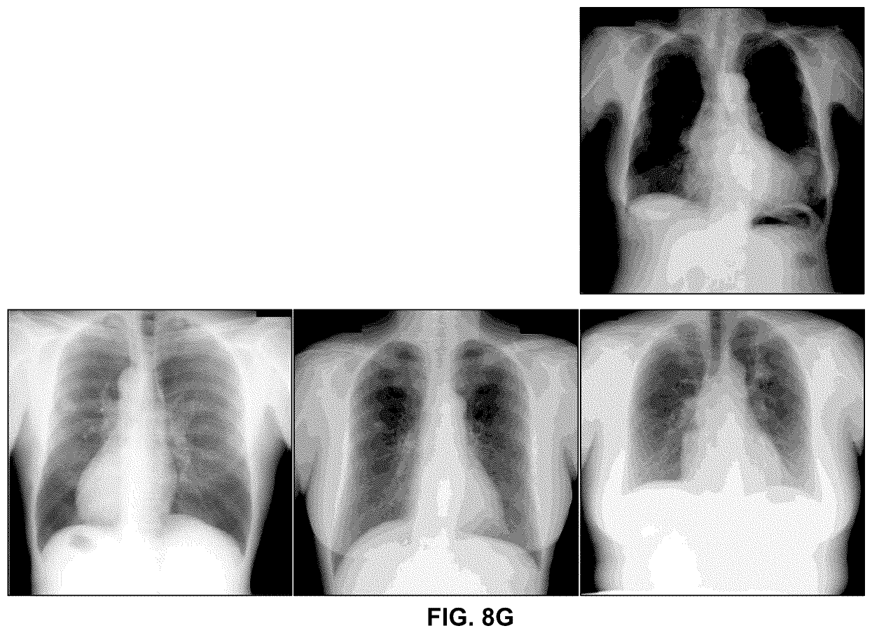

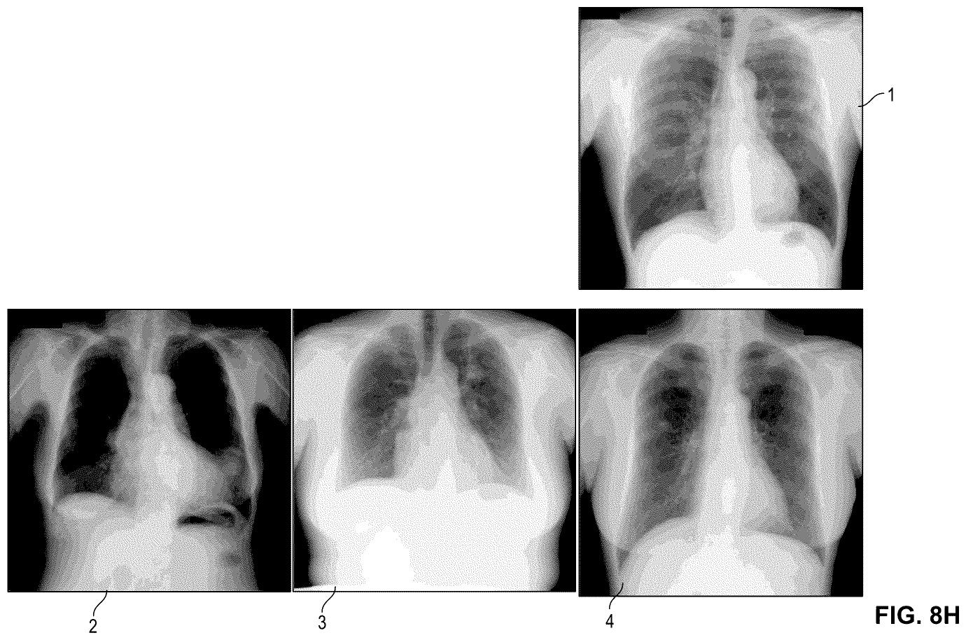

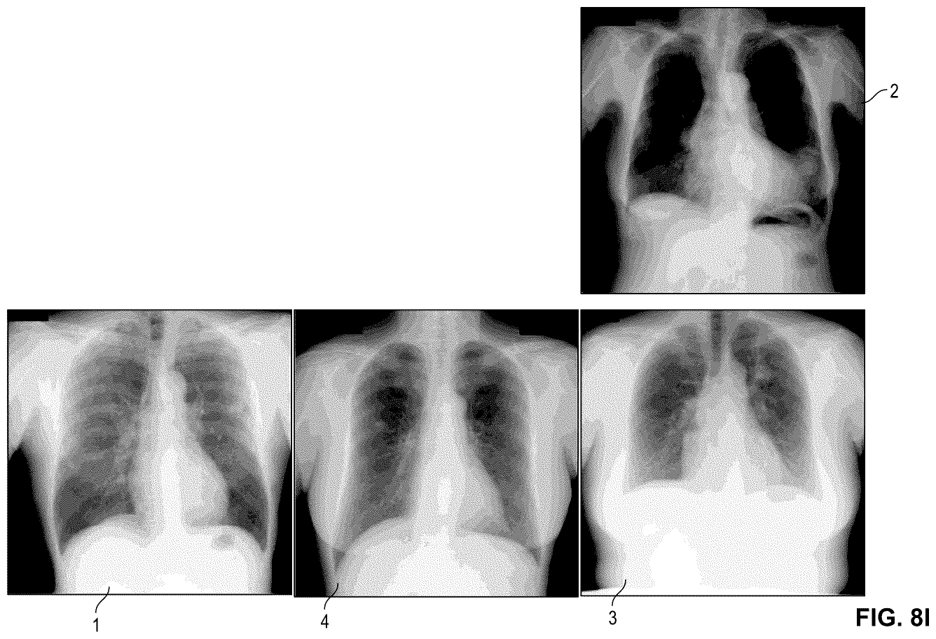

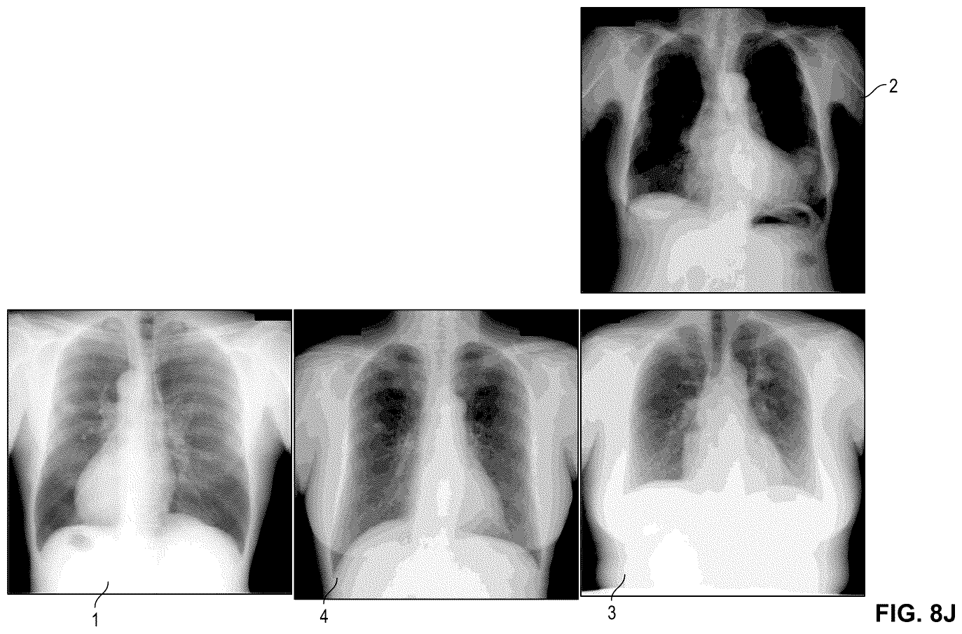

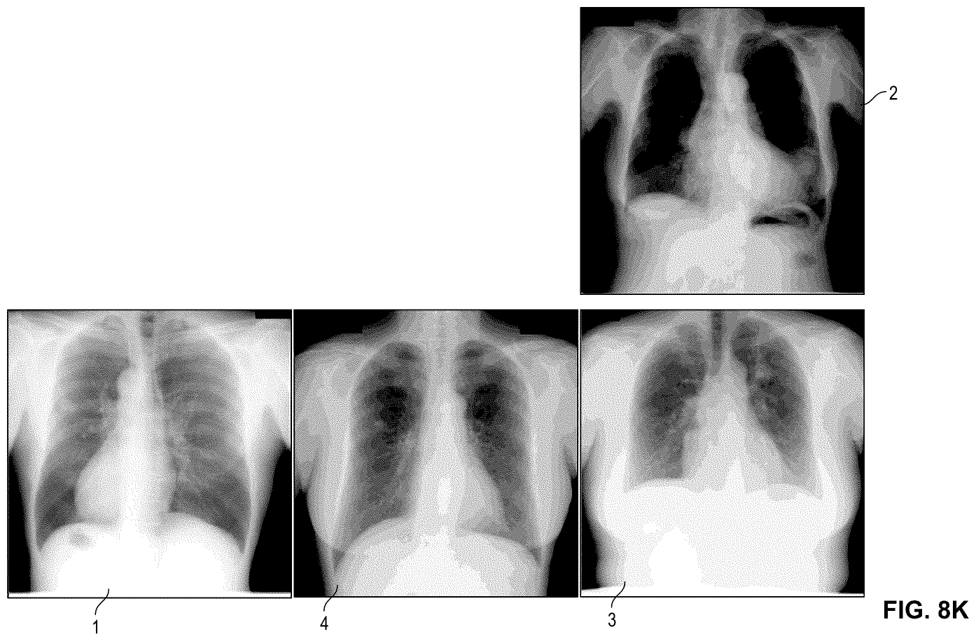

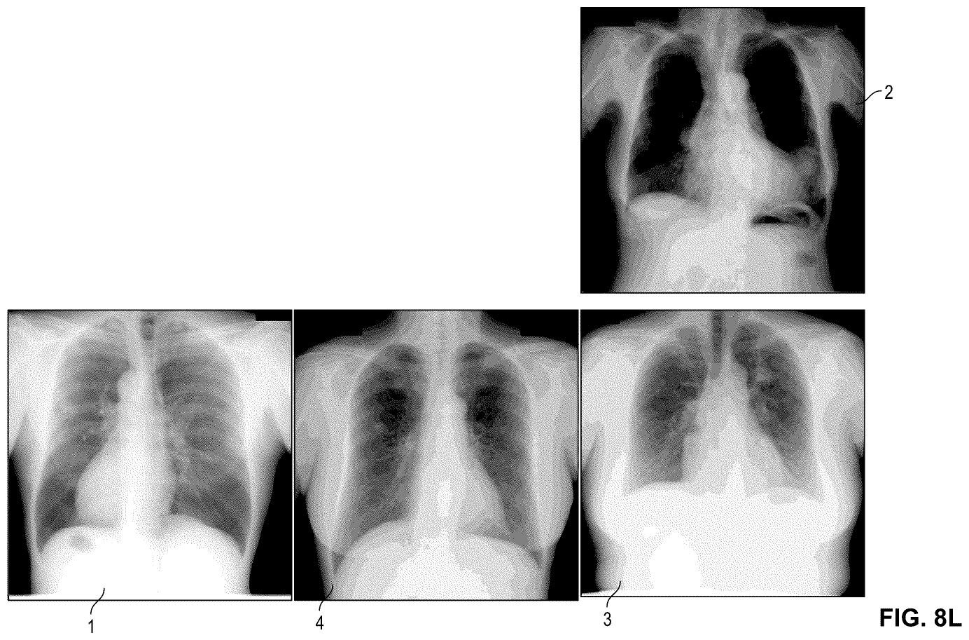

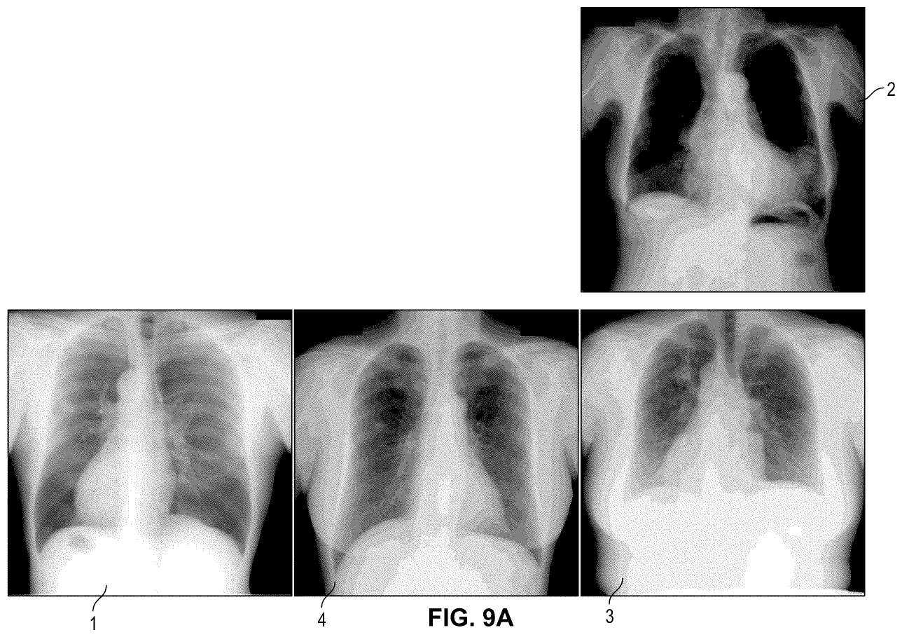

FIGS. 8A, 8B, 8C, 8D, 8E, 8F, 8G, 8H, 8I, 8J, 8K, and 8L depict example un-supervised learning models utilizing warm-up exercises. In particular, there is depicted the identification of varying unsupervised classifications as identified by the model as well as functions (e.g., github.com/openvinotoolkit/anomalib) for anomaly/novelty detection using autoencoder_img.zip via which to inject more regular chest X-rays (FIG. 8K) and functionality to establish dense (normal) anatomical embedding (FIG. 8L).

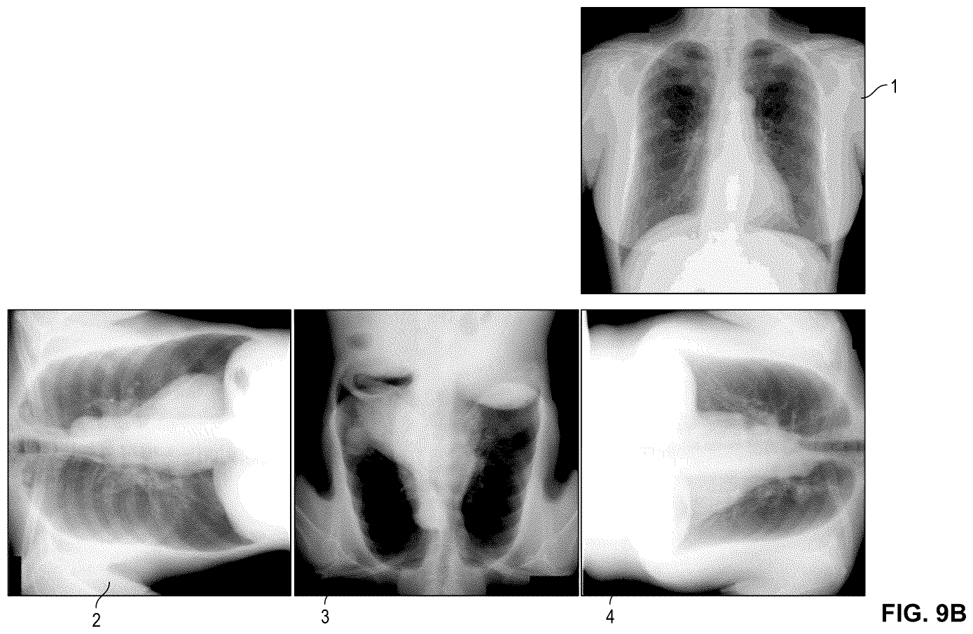

FIGS. 9A, and 9B depict example un-supervised learning models utilizing warm-up exercises. In particular, there is depicted the capability for unsupervised clustering via autoencoder_img.zip to determine balanced or imbalanced variants (FIG. 9A) in which images 1 and 3 are flipped about a vertical axis, and unsupervised clustering via directions01.zip to determine the presence of balanced or imbalanced orientations (FIG. 9B) such an up orientation in image 1, left in image 2, down in image 3 and right in image 4.

Using un-supervised learning models, anomaly detection may be applied to correct for (unusual) flipped images or to establish clustering using any of the up, down, right, and left flipped images.

Thus, through the combination of both supervised learning and unsupervised learning, applied functions available include classify orientations functions such as up, down, left, and right; classify genders (races) functions such as male and female; estimate ages (heights/weights) functions to establish or estimate ranges between 16˜ 89; semantic lung segmentation functions to establish lung masks; instance organ segmentation functions to establish organ masks; localize organs functions to establish organ bounding boxes anomaly detection functions for (unusual) flips, and clustering functions to carry out up, down, right, left, flips. Using function integration, a model may be designed and trained as one single model to accomplish all these tasks/functions, according to embodiments of the invention.

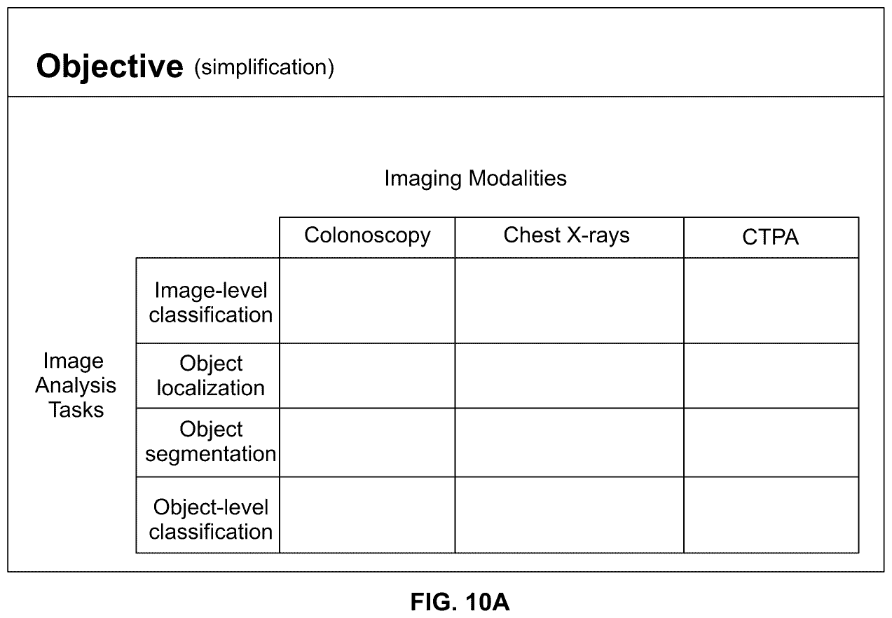

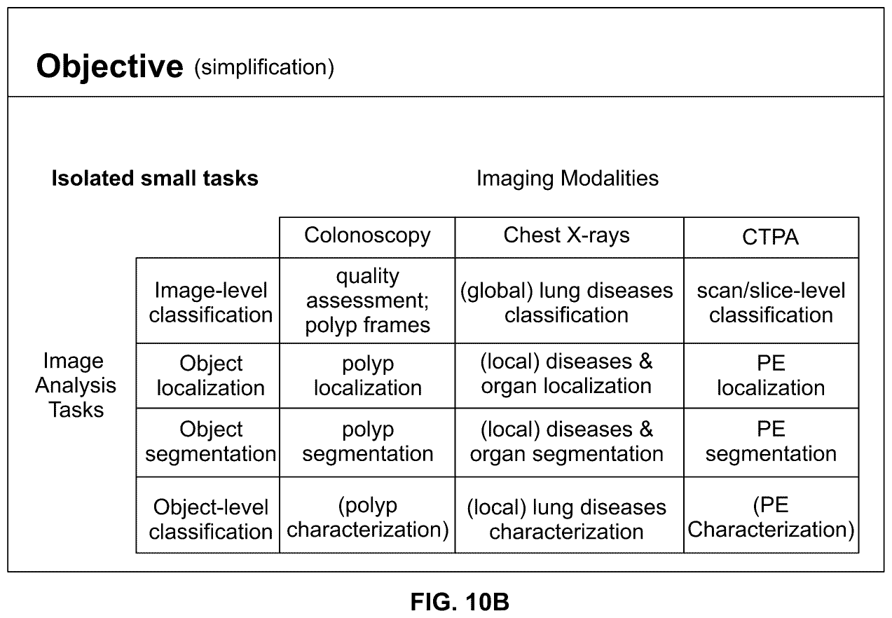

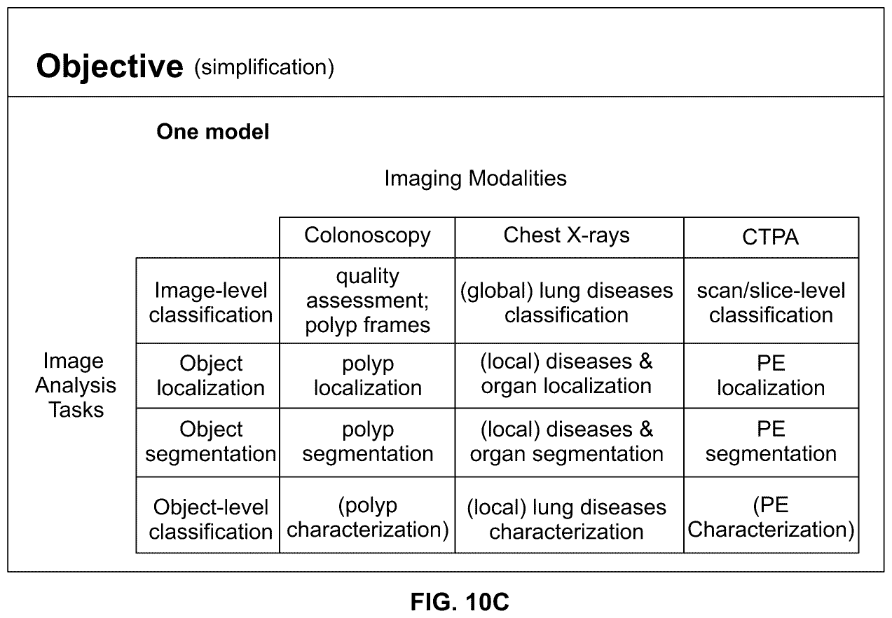

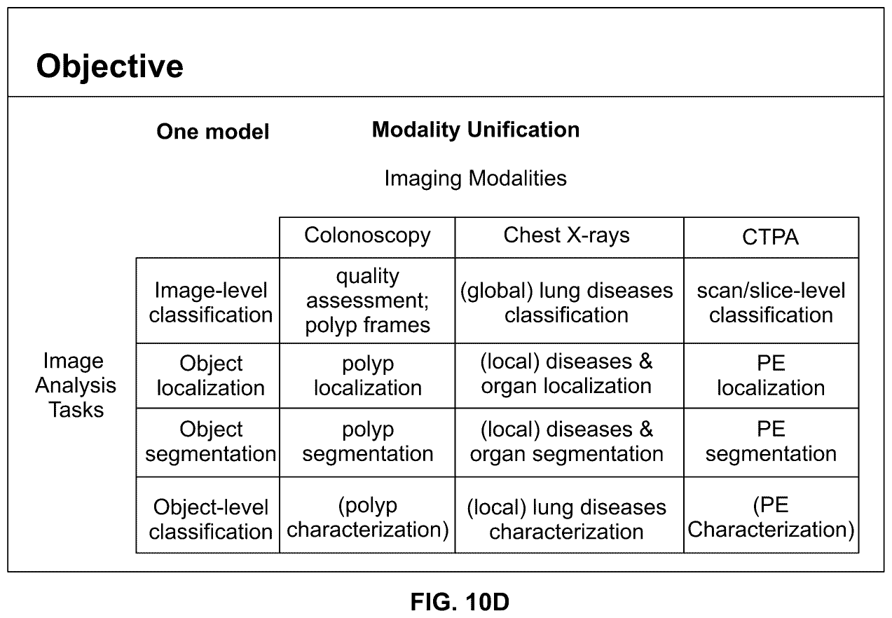

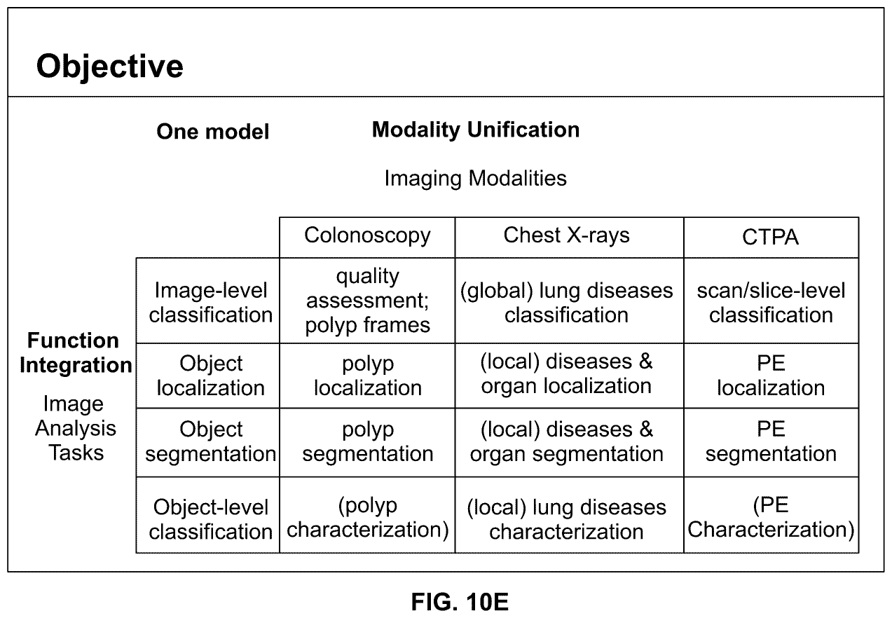

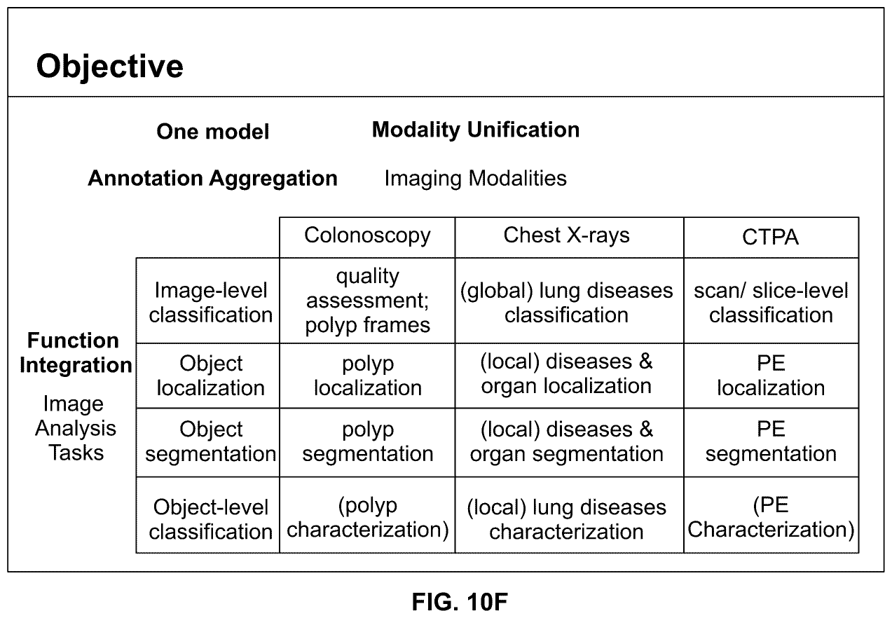

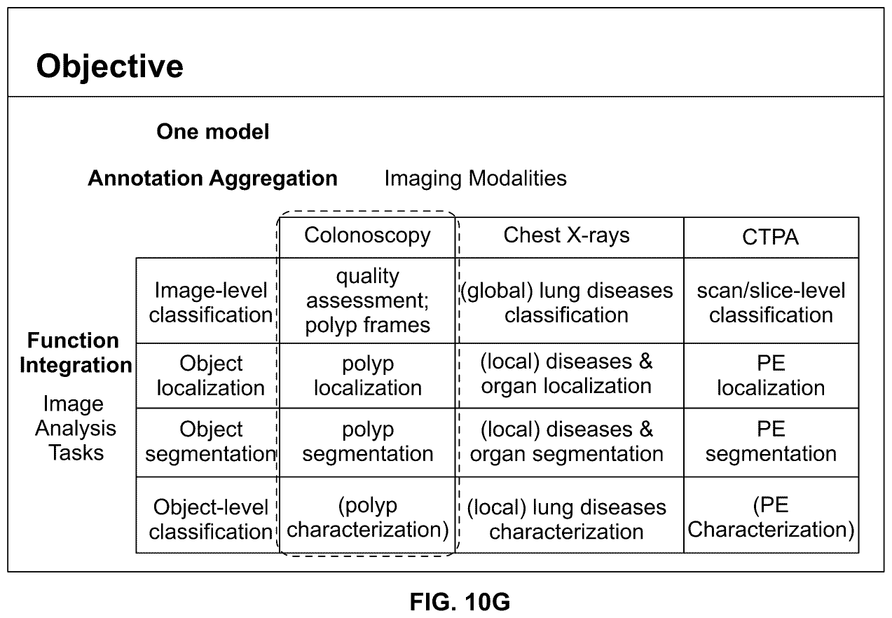

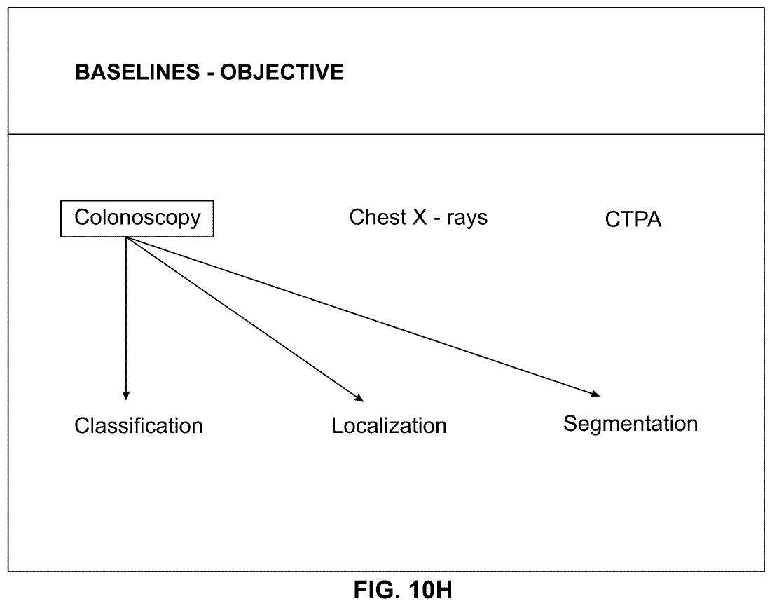

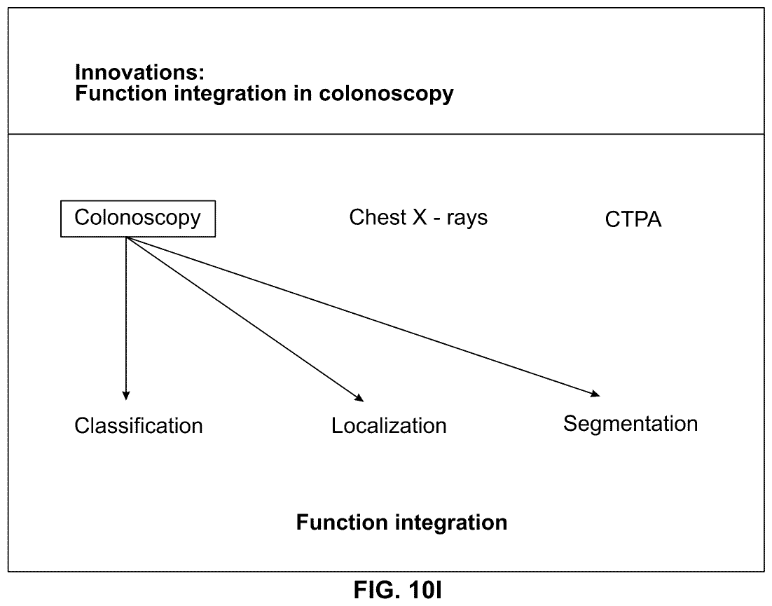

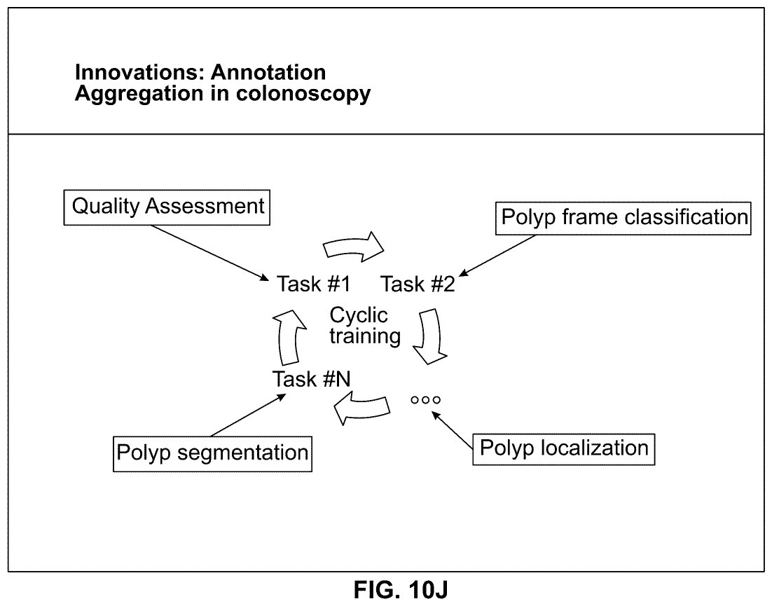

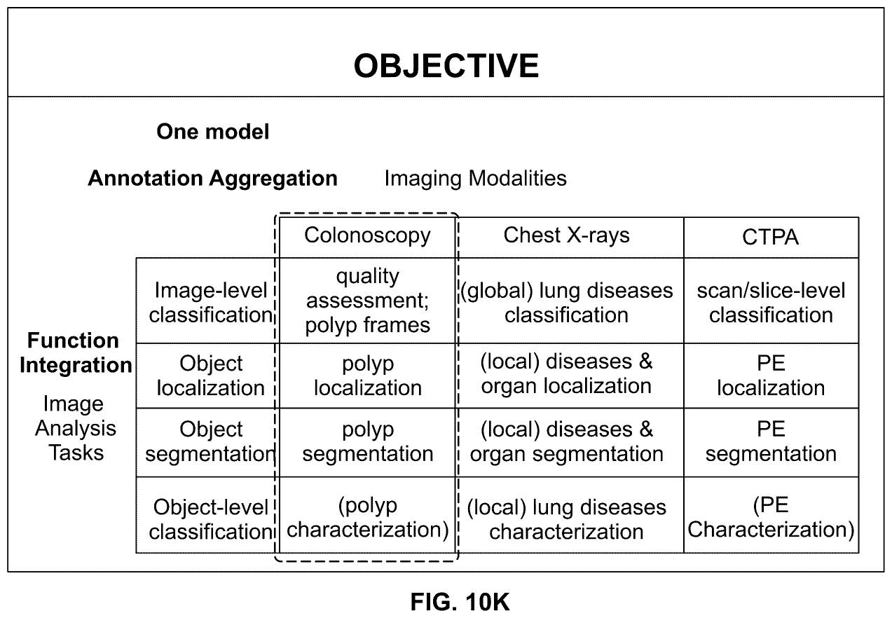

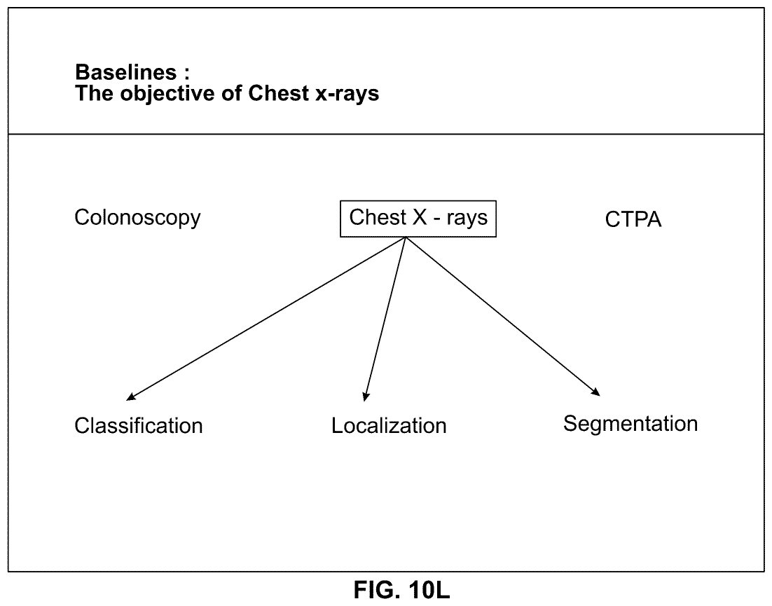

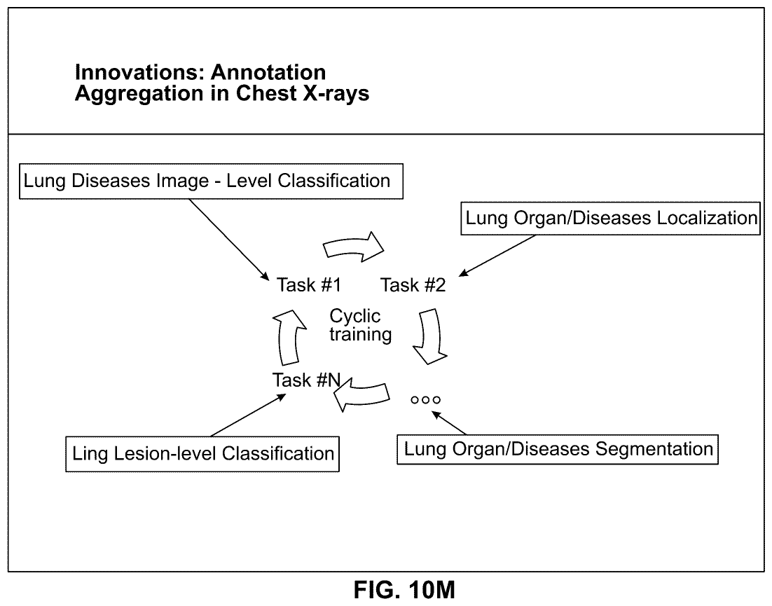

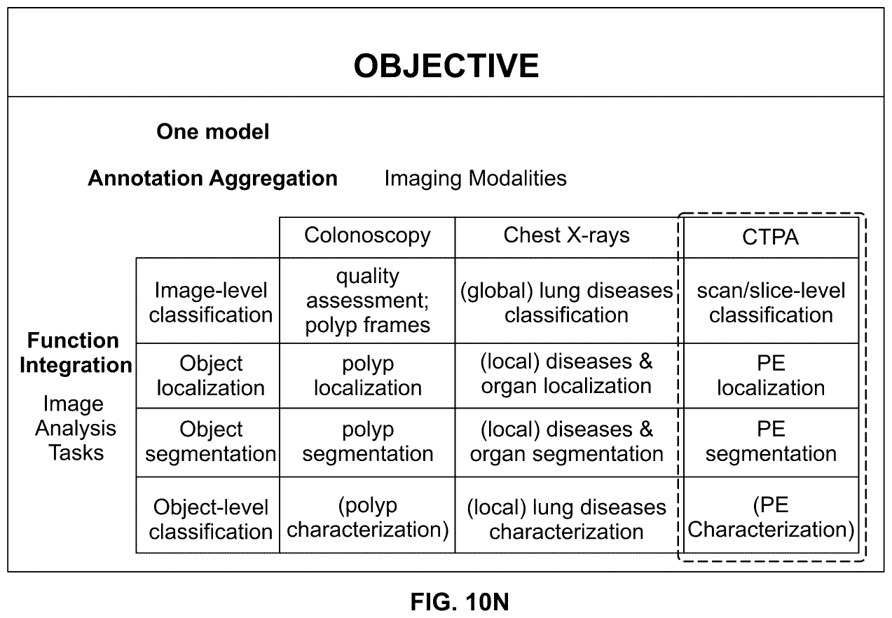

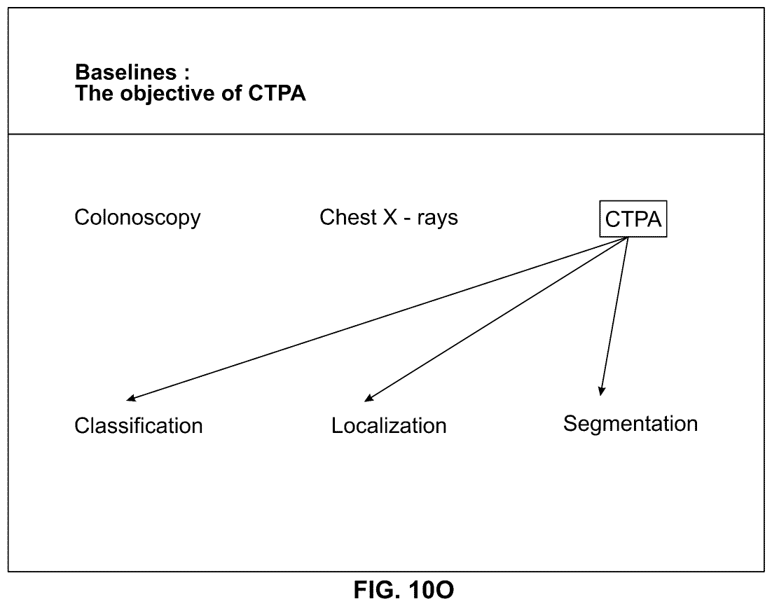

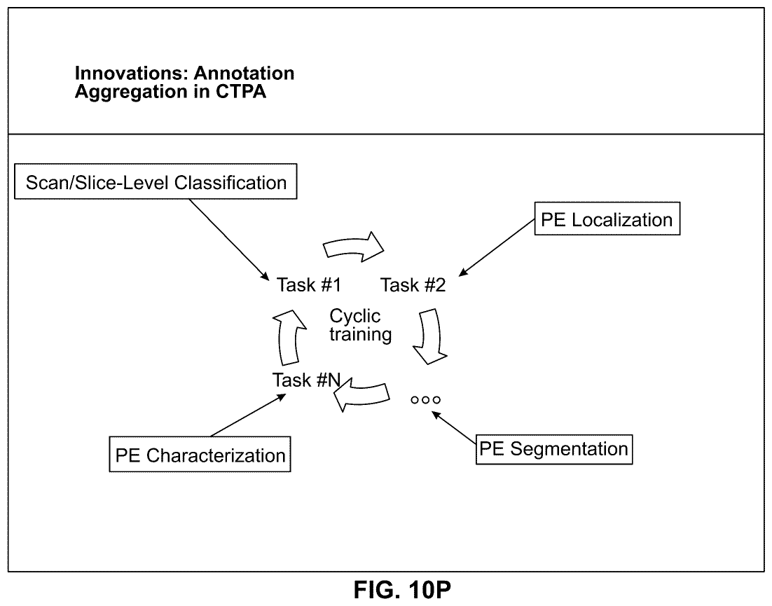

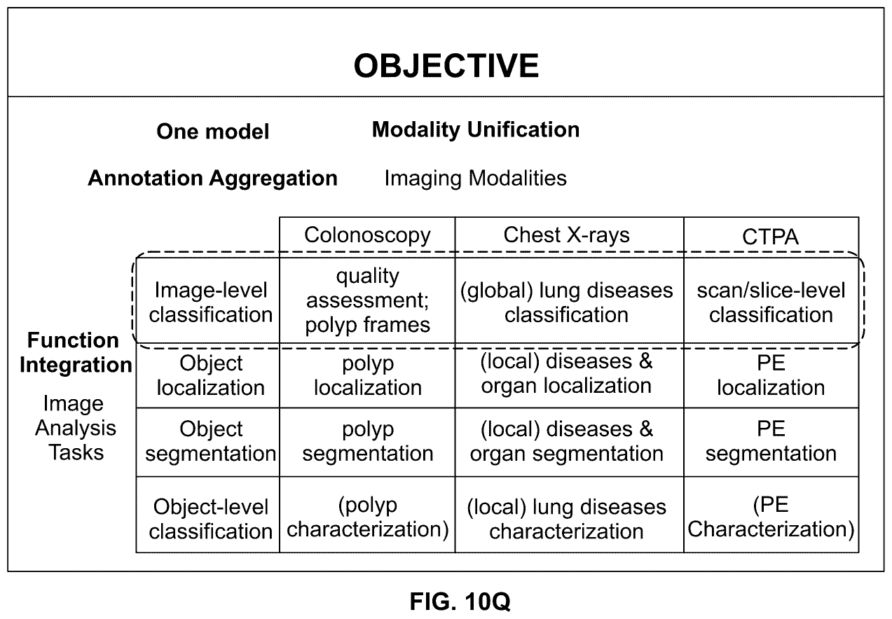

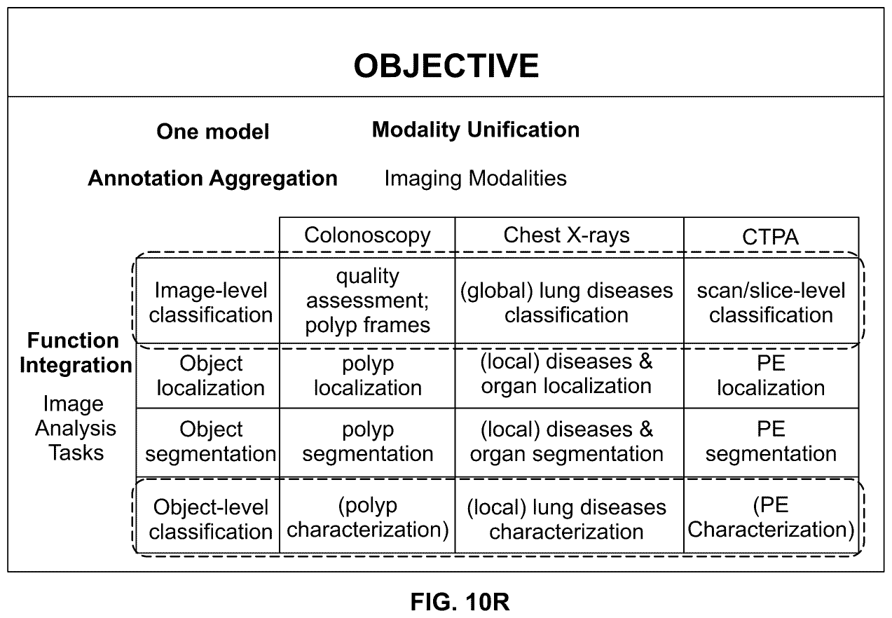

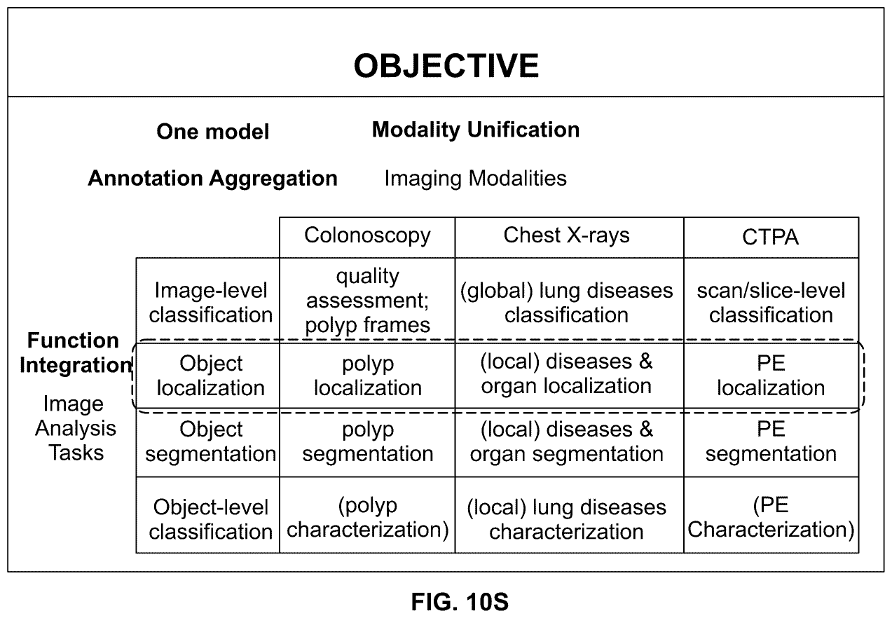

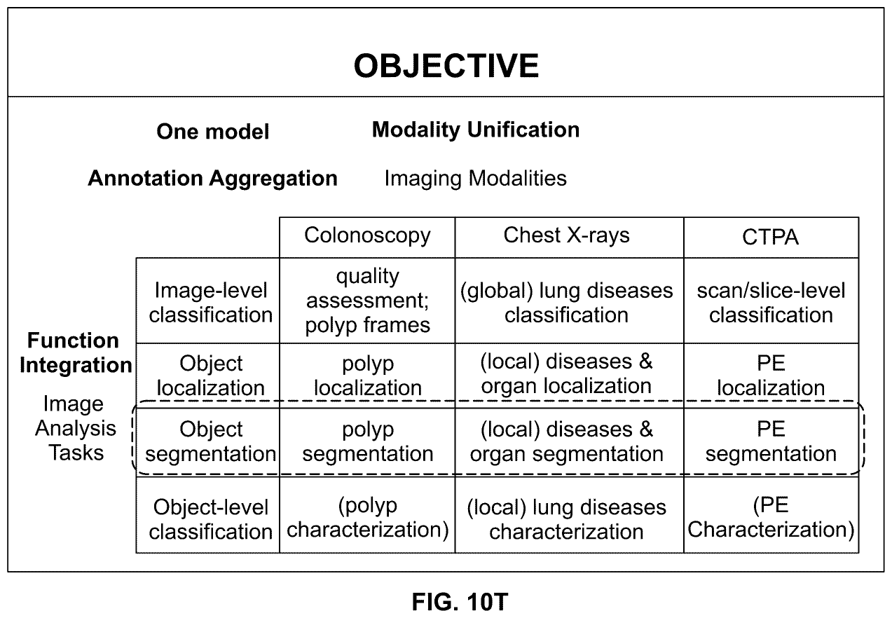

To that end, FIGS. 10A, 10B, 10C, 10D, 10E, 10F, 10G 10H, 10I, 10J, 10K, 10L, 10M, 10N, 10O, 10P, 10Q, 10R, 10S, and 10T depict various model integration objectives. Specifically shown here are objectives for rendering one model capable of multiple imaging modalities for multiple image analysis tasks, via function integration, and modality unification.

The disclosed methodologies create a novel method that can utilize many (different) image datasets and their associated heterogeneous annotations for classification, localization, and segmentation across imaging modalities to pre-train generic source models that are more robust, generalizable, and transferable to application-specific target tasks. More simply, the disclosed methodologies create a novel method to train a deep model that can aggregate different annotations and integrate functions of classification, localization, and segmentation for medical image analysis.

As shown here, the resulting model provides unified modalities across Colonoscopy, Chest X-rays, and CTPA and provides integrated functions across multiple image analysis tasks including image-level classification, object localization, object segmentation, and object-level classification tasks.

As depicted by FIG. 10G, an initial focus on colonoscopy, is to establish baselines for colonoscopy analysis, including each of classification, localization, and segmentation (FIG. 10H).

For instance, example embodiments begin to train a model to classify colonoscopy frame quality, using the ASU-Mayo Quality Assessment dataset with ResNet and Swin model Transformers.

Next, example embodiments train a model to localize polyps in colonoscopy using ASU-Mayo colonoscopy video dataset which via Faster, R-CNN, Mask R-CNN, and Swin Transformers.

Then example embodiments train a model to segment polyps in colonoscopy using ASU-Mayo colonoscopy video dataset via U-Net/UNet++ and Swin Transformers.

Then example embodiments proceed to integrate the various functions. Specifically, through the design of a new deep model integrating three functions and image classification quality assessment: (polyp frames), with object localization (polyp detection), object segmentation (polyp segmentation), and object classification (polyp types). Then example embodiments train the new model integrating partial annotations using ASU-Mayo quality assessment dataset, the ASU-Mayo colonoscopy video dataset, and all publicly or privately available datasets, including CVC-ClinicDB, ETIS-Larib, LDPolyp Video, and HyperKvasir.

For instance, for function integration in colonoscopy: example embodiments train the classification branch only, and specifically for polyp segmentation as the baseline. Then example embodiments train the classification branch for polyp frame classification jointly with the U-Net branch for polyp segmentation as the baseline, then re-use the pre-trained classification branch from before rather than training from scratch, then re-use the pre-trained U-Net branch from before rather than training from scratch by averaging the weights shared by both branches. Next example embodiments compare the performance of the models trained in the prior steps with the baselines. Next example embodiments replace the TransVW model with UperNet with Swin as the backbone and repeat the prior operations for evaluation. Then example embodiments extend the established models by including the frame quality assessment and then extend further by including the polyp localization, and finally compare results.

Again, example embodiments integrate the various functions for colonoscopy, specifically integrating classification, localization, and segmentation (FIG. 10I). Example embodiments apply the annotation aggregation flow for colonoscopy, starting with task 1, quality assessment, followed by task 2, polyp frame classification, then task . . . , polyp localization, and finally task N, polyp segmentation, as a cycle or loop (FIG. 10J).

The next focus, according to example embodiments, as depicted by FIG. 10K, is to focus on chest X-rays and to establish baselines for chest X-ray analysis, including each of classification, localization, and segmentation (FIG. 10L).

Next, example embodiments train a model to classify common lung diseases using chestXRays14, CheXpert, MIMIC-III.

Then example embodiments train a model to localize lung diseases using VinDr CXR, Faster R-CNN, Mask R-CNN, and Swin Transformers.

Next, example embodiments train a model to segment lung diseases/organs using JSRT and Pneumothorax, U-Net/U-Net++, and Swin Transformers.

Then example embodiments proceed to integrate the various functions. Specifically, through the design a new deep model integrating three functions and image classification of lung diseases: image-level labels, object localization (lung diseases: bounding-box labels), object segmentation (organ/diseases segmentation), and object classification (characterization and prognosis). Then example embodiments train the new model integrating partial annotations using all publicly or privately available datasets, including ChestXRay14, CheXpert, SIIM-ACR Pneumothorax Segmentation, RSNA Pneumonia Detection Challenge, VinDr CXR, MIMIC-III, Shenzhen, and PadChest. Then example embodiments again apply the annotation aggregation cycle training loop, for the chest X-rays (FIG. 10M), starting with task 1, lung diseases image-level classification, followed by task 2, lung organ/diseases localization, then task . . . , lung organ/diseases segmentation, and finally task N, lung organ/diseases classification, as a cycle or loop.

Shifting focus to analysis of chest CT (FIG. 10N) and specifically “CTPA” (Computed Tomography Pulmonary Angiogram), a novel method is provided to train a deep model that can aggregate different annotations and integrate functions of classification, localization, and segmentation for CTPA (CT Pulmonary Angiogram), according to embodiments of the invention.

As depicted by FIG. 10O, with a new focus on CTPA, the objective according to embodiments is to establish baselines for CTPA analysis, including each of classification, localization, and segmentation (FIG. 10O).

For instance, example embodiments begin to train a model to classify PE slices/candidates, using RSNA PE, ResNet, and Swin Transformers.

Next, example embodiments train a model to localize PE using the CAD PE dataset, FUMPE, Faster R-CNN, Mask R-CNN, and Swin Transformers.

Then example embodiments train a model to segment PE and lungs using the CAD PE dataset, FUMPE, U-Net/U-Net++, and Swin Transformers.

Then example embodiments proceed to integrate the various functions. Specifically, through the design a new deep model is provided, integrating three functions and image/scan classification slice/scan-level labels); with object localization (PE: centroids or bounding boxes); object segmentation (lung/PE segmentation); and object classification (PE characterization, prognosis). Then example embodiments train the new model integrating partial annotations using ASU dataset, and all publicly or privately available datasets, including RSNA PE, CAD PE Challenge, an FUMPE.

Once again, example embodiments integrate the various functions for CTPA, specifically integrating classification, localization, and segmentation. Example embodiments apply the annotation aggregation flow for CTPA, starting with task 1, scan/slice-level classification, followed by task 2, PE localization, then task . . . , PE Segmentation, and finally task N, PE Characterization, as a cycle or loop (FIG. 10P).

The next objective, according to embodiments of the invention, at FIG. 10Q, is modality unification for classification through the development of a novel method to train a deep model that can aggregate different annotations for classification across different modalities, including colonoscopy, chest X-rays, and CTPA.

With a focus on the localization function, the objective is further to develop a novel method to train a deep model that can aggregate different annotations for localization across different modalities, including colonoscopy, chest X-rays, and CTPA.

Next with a focus on the segmentation function, the objective is further to develop a novel method to train a deep model that can aggregate different annotations for segmentation across different modalities, including colonoscopy, chest X-rays, and CTPA.

Then, with a focus each of function integration and modality integration with annotation aggregation together, the objective is to create a novel method that can utilize many (different) image datasets and their associated heterogeneous annotations for classification, localization, and segmentation across imaging modalities to pre-train generic source models that are more robust, generalizable, and transferable to application-specific target tasks.

The effort, as it relates to Colonoscopy, includes:

First, (1) establish the SoTA function baselines in Colonoscopy for (a) frame quality assessment with ASU-Mayo Quality Assessment dataset (b) Polyp frame classification (using an ASU-Mayo Colonoscopy Video dataset, CVCClinicDB, LDPolyp Video, HyperKvasir, and PolypGen) (c) Polyp localization (using am ASU-Mayo Colonoscopy Video dataset, CVC-ClinicDB, LDPolypVideo, HyperKvasir, and PolypGen) (d) Polyp segmentation (using an ASU-Mayo Colonoscopy Video dataset, CVC-ClinicDB, LDPolypVideo, HyperKvasir, and PolypGen).

Next, (2) perform function integration in colonoscopy according to the described embodiments and finally, (3) demonstrate the benefits of function integration as superior over all other established baselines.

The effort, as it relates to Chest X-rays, includes:

First, (1) establish the SoTA function baselines in Chest X-rays for (a) Lung diseases classification with chestX-ray14 and chestXpert (b) Lung diseases localization with VinDr CXR (c) localization using VinDR-SpineXR (d) Segmentation of the lungs, heart, and clavicles using JSRT X-rays (e) Segmentation of pneumothorax using SIIM-ACR Pneumothorax Segmentation dataset (f) Segmentation of Ribs using VinDR-RibCXR.

Next, (2) perform function integration in chest X-rays according to the described embodiments relating to the function integration practice in Colonoscopy.

Finally, (3) demonstrate the benefits of function integration over all other established baselines.

The effort, as it relates to CTPA, includes:

First, (1) establish the SoTA function baselines in CTPA for (a) Slice-level PE classification using RSNA PE dataset (b) Scan-level classification using RSNA PE dataset (c) Clot-level classification in 3D using a PE dataset (d) PE colocalization using CAD PE and FUMPE datasets (e) PE segmentation using CAD PE and FUMPE datasets.

Next, (2) perform Function Integration in CTPA according to the described embodiments relating to the function integration practice in Colonoscopy.

Finally, (3) demonstrate the benefits of function integration over all otherbaselines

Establishing a Classification Function includes: (1) Establish the SoTA classification baselines in Colonoscopy (a) Frame quality assessment with an ASU-Mayo Quality Assessment dataset (b) polyp frame classification (using an ASU-Mayo Colonoscopy video dataset, CVCClinicDB, LDPolypVideo, HyperKvasir, and PolypGen) (2) Establish the SoTA classification baselines in Chest X-rays (a) Lung diseases classification with chestX-ray14 and chestXpert (3) Establish the SoTA classification baselines in CTPA (a) Slice-level PE classification using RSNA PE dataset (b) Scan-level classification using RSNA PE dataset (c) Clot-level classification in 3D using a PE dataset (4) Perform Modality Unification for classification: train one model that can perform classification across Colonoscopy, chest X-rays, and CTPA (5) Demonstrate the benefits of Modality Unification over all other classification baselines.

Establishing a Localization Function includes: (1) Establish the SoTA localization baselines in Colonoscopy (a) Polyp segmentation (using an ASU-Mayo Colonoscopy Video dataset, CVC-ClinicDB, LDPolypVideo, HyperKvasir, and PolypGen) (2) Establish the SoTA localization baselines in Chest X-rays (a) Lung diseases localization with VinDr CXR (3) Establish the SoTA localization baselines in CTPA (a) PE colocalization using CAD PE and FUMPE datasets (4) Perform Modality Unification for localization: train one model that can perform localization across Colonoscopy, chest X-rays, and CTPA (5) Demonstrate the benefits of modality unification over all other localization baselines.

Establishing a Segmentation Function includes: (1) Establish the SoTA segmentation baselines in Colonoscopy (a) Polyp localization (using an ASU-Mayo Colonoscopy Video dataset, CVC-ClinicDB, LDPolypVideo, HyperKvasir, and PolypGen) (2) Establish the SoTA segmentation baselines in Chest X-rays (a) Segmentation of the lungs, heart, and clavicles using JSRT X-rays (b) Segmentation of pneumothorax using SIIM-ACR Pneumothorax Segmentation dataset (3) Establish the SoTA segmentation baselines in CTPA (a) PE segmentation using CAD PE and FUMPE datasets (4) Perform Modality Unification for segmentation: train one model that can perform segmentation across Colonoscopy, chest X-rays, and CTPA (5) Demonstrate the benefits of modality unification over all other localization baselines.

Establishing One Model for All, thus Achieving Modality Unification, Function Integration, and Annotation Aggregation includes: (1) Establish the SoTA function baselines in Colonoscopy for (a) Frame quality assessment with an ASU-Mayo Quality Assessment dataset (b) Polyp frame classification (using an ASU-Mayo Colonoscopy Video dataset, CVCClinicDB, LDPolypVideo, HyperKvasir, and PolypGen) (c) Polyp localization (using an ASU-Mayo Colonoscopy Video dataset, CVC-ClinicDB, LDPolypVideo, HyperKvasir, and PolypGen) (d) Polyp segmentation (using an ASU-Mayo Colonoscopy Video dataset, CVC-ClinicDB, LDPolypVideo, HyperKvasir, and PolypGen) (2) Establish the SoTA function baselines in Chest X-rays for (a) Lung diseases classification with chestX-ray14 and chestXpert (b) Lung diseases localization with VinDr CXR (c) Segmentation of the lungs, heart, and clavicles using JSRT X-rays (d) Segmentation of pneumothorax using SIIM-ACR Pneumothorax Segmentation dataset (3) Establish the SoTA function baselines in CTPA for (a) Slice-level PE classification using RSNA PE dataset (b) Scan-level classification using RSNA PE dataset (c) Clot-level classification in 3D using a PE dataset (d) PE colocalization using CAD PE and FUMPE datasets (e) PE segmentation using CAD PE and FUMPE datasets (4) Perform and demonstrate the benefits of Function Integration across Modalities (a) Colonoscopy (b) Chest X-rays (c) CTPA (5) Perform and demonstrate the benefits of Modality Unification across Functions (a) Classification (b) Localization (c) Segmentation (6) Perform and demonstrate the benefits of both Modality Unification and Function Integration.

### CONCLUSION

Embodiments include various operations. The operations described in accordance with such embodiments may be performed by hardware components or may be embodied in machine-executable instructions, which may be used to cause a specialized and special-purpose processor having been programmed with the instructions to perform the operations described herein. Alternatively, the operations may be performed by a combination of hardware and software. In such a way, the embodiments of the invention provide a technical solution to a technical problem.

Embodiments also relate to an apparatus for performing the operations disclosed herein. This apparatus may be specially constructed for the required purposes, or it may be a special purpose computer selectively activated or reconfigured by a computer program stored in the computer. Such a computer program may be stored in a computer readable storage medium, such as, but not limited to, any type of disk including floppy disks, optical disks, CD-ROMs, and magnetic-optical disks, read-only memories (ROMs), random access memories (RAMs), EPROMS, EEPROMs, magnetic or optical cards, or any type of media suitable for storing electronic instructions, each coupled to a computer system bus.

While the algorithms and displays presented herein are not inherently related to any particular computer or other apparatus, they are specially configured and implemented via customized and specialized computing hardware which is specifically adapted to more effectively execute the novel algorithms and displays. Various customizable and special purpose systems may be utilized in conjunction with specially configured programs in accordance with the teachings herein, or it may prove convenient, in certain instances, to construct a more specialized apparatus to perform the required method steps. In addition, embodiments are not described with reference to any particular programming language. It will be appreciated that a variety of programming languages may be used to implement the teachings of the embodiments as described herein.

Embodiments may be provided as a computer program product, or software, that may include a machine-readable medium having stored thereon instructions, which may be used to program a computer system (or other electronic devices) to perform a process according to the disclosed embodiments. A machine-readable medium includes any mechanism for storing or transmitting information in a form readable by a machine (e.g., a computer). For example, a machine-readable (e.g., computer-readable) medium includes a machine (e.g., a computer) readable storage medium (e.g., read only memory (“ROM”), random access memory (“RAM”), magnetic disk storage media, optical storage media, flash memory devices, etc.), a machine (e.g., computer) readable transmission medium (electrical, optical, acoustical), etc.

Any of the disclosed embodiments may be used alone or together with one another in any combination. Although various embodiments may have been partially motivated by deficiencies with conventional techniques and approaches, some of which are described or alluded to within the specification, the embodiments need not necessarily address or solve any of these deficiencies, but rather, may address only some of the deficiencies, address none of the deficiencies, or be directed toward different deficiencies and problems which are not directly discussed.

It is appreciated that a machine in the exemplary form of a computer system, in accordance with one embodiment, includes a set of instructions that may be executed to cause the machine/computer system to perform any one or more of the methodologies discussed herein.

In alternative embodiments, the machine may be connected (e.g., networked) to other machines in a Local Area Network (LAN), an intranet, an extranet, or the public Internet. The machine may operate in the capacity of a server or a client machine in a client-server network environment, as a peer machine in a peer-to-peer (or distributed) network environment, as a server or series of servers within an on-demand service environment. Certain embodiments of the machine may be in the form of a personal computer (PC), a tablet PC, a set top box (STB), a Personal Digital Assistant (PDA), a cellular telephone, a web appliance, a server, a network router, switch or bridge, computing system, or any machine capable of executing a set of instructions (sequential or otherwise) that specify and mandate the specifically configured actions to be taken by that machine pursuant to stored instructions. Further, while the machine may be a single machine, the term “machine” shall also be taken to include any collection of machines (e.g., computers) that individually or jointly execute a set (or multiple sets) of instructions to perform any one or more of the methodologies discussed herein.

An exemplary computer system includes a processor, a main memory (e.g., read-only memory (ROM), flash memory, dynamic random access memory (DRAM) such as synchronous DRAM (SDRAM) or Rambus DRAM (RDRAM), etc., static memory such as flash memory, static random access memory (SRAM), volatile but high-data rate RAM, etc.), and a secondary memory (e.g., a persistent storage device including hard disk drives and a persistent database and/or a multi-tenant database implementation), which communicate with each other via a bus. Main memory includes an encoder-decoder network (e.g., such as an encoder-decoder implemented via a neural network model) for performing operations including processing medical imaging in support of the methodologies and techniques described herein. Main memory and its sub-elements are further operable in conjunction with processing logic and processor to perform the methodologies discussed herein.

Processor represents one or more specialized and specifically configured processing devices such as a microprocessor, central processing unit, or the like. More particularly, the processor may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processor may also be one or more special-purpose processing devices such as an application-specific integrated circuit (ASIC), a field programmable gate array (FPGA), a digital signal processor (DSP), network processor, or the like. The processor is configured to execute the processing logic for performing the operations and functionality discussed herein.

The computer system may further include a network interface card. The computer system also may include a user interface (such as a video display unit, a liquid crystal display, etc.), an alphanumeric input device (e.g., a keyboard), a cursor control device (e.g., a mouse), and a signal generation device (e.g., an integrated speaker). The computer system may further include peripheral device (e.g., wireless or wired communication devices, memory devices, storage devices, audio processing devices, video processing devices, etc.).

The secondary memory may include a non-transitory machine-readable storage medium or a non-transitory computer readable storage medium or a non-transitory machine-accessible storage medium on which is stored one or more sets of instructions (e.g., software) embodying any one or more of the methodologies or functions described herein. The software may also reside, completely or at least partially, within the main memory and/or within the processor during execution thereof by the computer system, the main memory and the processor also constituting machine-readable storage media. The software may further be transmitted or received over a network via the network interface card.

Thus, embodiments include a system that has a memory to store instructions, and a processor to execute the instructions stored in the memory. The system is configured to execute instructions for implementing a unified AI model pre-trained for use with medical image classification, medical image localization, and medical image segmentation, in the context of medical image analysis, by performing the following operations: receiving medical image data at the system from a plurality of datasets provided via publicly or privately available sources; training the AI model on the datasets to learn image classification and output (i) an image-level classification function, (ii) an object-level classification function, and (iii) a plurality of image classification weights; training the AI model on the datasets to learn image localization and output an object localization function and a plurality of image localization weights; training the AI model on the datasets to learn image segmentation and output an object segmentation function and a plurality of image segmentation weights; integrating each of the image classification weights, the image localization weights, and the image segmentation weights into a single pre-trained AI model; integrating each of the image-level classification function, the object-level classification function, the object localization function and the object segmentation function into the single pre-trained AI model; and outputting the pre-trained AI model for use with medical image analysis.

According to some embodiments, receiving the medical image data at the system further comprises receiving a plurality of private datasets provided via non-public sources.

According to some embodiments, training the AI model on the datasets comprises executing unsupervised learning operations on the datasets via the AI model.

According to some embodiments, training the AI model on the datasets comprises executing supervised learning operations on the datasets via the AI model.

According to some embodiments, training the AI model on the datasets comprises executing deep learning operations on the datasets via the AI model.

According to some embodiments, training the AI model on the datasets comprises training generic source models having strong generalizability and transferability to yield application-specific target models having superior task performance in the target task.

According to some embodiments, training the AI model on the datasets comprises training the AI model to generate as its output one or more of: a prediction of disease in a medical image; a prediction of no disease in a medical image; an image-level label not present in the source image; an organ or lesion marker not present in the source image; an organ or lesion bounding box not present in the source image; and an organ or lesion mask not present in the source image.

While the subject matter disclosed herein has been described by way of example and in terms of the specific embodiments, it is to be understood that the claimed embodiments are not limited to the explicitly enumerated embodiments disclosed. To the contrary, the disclosure is intended to cover various modifications and similar arrangements as are apparent to those skilled in the art. Therefore, the scope of the appended claims is to be accorded the broadest interpretation to encompass all such modifications and similar arrangements. It is to be understood that the above description is intended to be illustrative, and not restrictive. Many other embodiments will be apparent to those of skill in the art upon reading and understanding the above description. The scope of the disclosed subject matter is therefore to be determined in reference to the appended claims, along with the full scope of equivalents to which such claims are entitled.

### Systems, Methods, and Apparatuses for Implementing Improved Generalizability, Transferability, and Robustness Through Modality Unification, Function Integration, and Annotation Aggregation

What is claimed is:

1. A system comprising:
a memory to store instructions;
a processor to execute the instructions stored in the memory;
wherein the system is specially configured to execute instructions for implementing a unified AI model pre-trained for use with medical image classification, medical image localization, and medical image segmentation, in the context of medical image analysis, by performing the following operations:
receiving medical image data at the system from a plurality of datasets provided via publicly or privately available sources;
training the AI model on the datasets to learn image classification and output (i) an image-level classification function, (ii) an object-level classification function, and (iii) a plurality of image classification weights;
training the AI model on the datasets to learn image localization and output an object localization function and a plurality of image localization weights;
training the AI model on the datasets to learn image segmentation and output an object segmentation function and a plurality of image segmentation weights;
integrating each of the image classification weights, the image localization weights, and the image segmentation weights into a single pre-trained AI model;
integrating each of the image-level classification function, the object-level classification function, the object localization function and the object segmentation function into the single pre-trained AI model; and
outputting the pre-trained AI model for use with medical image analysis.

2. The system of claim 1, wherein receiving the medical image data at the system further comprises receiving a plurality of private datasets provided via non-public sources.

3. The system of claim 1, wherein training the AI model on the datasets comprises executing unsupervised learning operations on the datasets via the AI model.

4. The system of claim 1, wherein training the AI model on the datasets comprises executing supervised learning operations on the datasets via the AI model.

5. The system of claim 1, wherein training the AI model on the datasets comprises executing deep learning operations on the datasets via the AI model.

6. The system of claim 1, wherein training the AI model on the datasets comprises training generic source models having strong generalizability and transferability to yield application-specific target models having superior task performance in the target task.

7. The system of claim 1, wherein training the AI model on the datasets comprises training the AI model to generate as its output, one or more of:
a prediction of disease in a medical image;
a prediction of no disease in a medical image;
an image-level label not present in the source image;
an organ or lesion marker not present in the source image;
an organ or lesion bounding box not present in the source image; and
an organ or lesion mask not present in the source image.

8. A computer-implemented method performed by a system having at least a processor and a memory therein to execute instructions for implementing a unified AI model pre-trained for use with medical image classification, medical image localization, and medical image segmentation, in the context of medical image analysis, wherein the method comprises:
receiving medical image data at the system from a plurality of datasets provided via publicly or privately available sources;
training the AI model on the datasets to learn image classification and output (i) an image-level classification function, (ii) an object-level classification function, and (iii) a plurality of image classification weights;
training the AI model on the datasets to learn image localization and output an object localization function and a plurality of image localization weights;
training the AI model on the datasets to learn image segmentation and output an object segmentation function and a plurality of image segmentation weights;
integrating each of the image classification weights, the image localization weights, and the image segmentation weights into a single pre-trained AI model;
integrating each of the image-level classification function, the object-level classification function, the object localization function and the object segmentation function into the single pre-trained AI model; and
outputting the pre-trained AI model for use with medical image analysis.

9. The computer-implemented method of claim 8, wherein the receiving the medical image data at the system further comprises receiving a plurality of private datasets provided via non-public sources.

10. The computer-implemented method of claim 8, wherein the training the AI model on the datasets comprises executing unsupervised learning operations on the datasets via the AI model.

11. The computer-implemented method of claim 8, wherein the training the AI model on the datasets comprises executing supervised learning operations on the datasets via the AI model.

12. The computer-implemented method of claim 8, wherein the training the AI model on the datasets comprises executing deep learning operations on the datasets via the AI model.

13. The computer-implemented method of claim 8, wherein the training the AI model on the datasets comprises training generic source models having strong generalizability and transferability to yield application-specific target models having superior task performance in the target task.

14. The computer-implemented method of claim 8, wherein the training the AI model on the datasets comprises training the AI model to generate as its output, one or more of:
a prediction of disease in a medical image;
a prediction of no disease in a medical image;
an image-level label not present in the source image;
an organ or lesion marker not present in the source image;
an organ or lesion bounding box not present in the source image; and
an organ or lesion mask not present in the source image.

15. Non-transitory computer readable storage media having instructions stored thereupon that, when executed by a system having at least a processor and a memory therein, the instructions cause the processor to execute instructions for implementing a unified AI model pre-trained for use with medical image classification, medical image localization, and medical image segmentation, in the context of medical image analysis, by performing the following operations:
receiving medical image data at the system from a plurality of datasets provided via publicly or privately available sources;
training the AI model on the datasets to learn image classification and output (i) an image-level classification function, (ii) an object-level classification function, and (iii) a plurality of image classification weights;
training the AI model on the datasets to learn image localization and output an object localization function and a plurality of image localization weights;
training the AI model on the datasets to learn image segmentation and output an object segmentation function and a plurality of image segmentation weights;
integrating each of the image classification weights, the image localization weights, and the image segmentation weights into a single pre-trained AI model;
integrating each of the image-level classification function, the object-level classification function, the object localization function and the object segmentation function into the single pre-trained AI model; and
outputting the pre-trained AI model for use with medical image analysis.

16. The non-transitory computer readable storage media of claim 15, wherein receiving the medical image data at the system further comprises receiving a plurality of private datasets provided via non-public sources.

17. The non-transitory computer readable storage media of claim 15, wherein training the AI model on the datasets comprises executing unsupervised learning operations on the datasets via the AI model.

18. The non-transitory computer readable storage media of claim 15, wherein training the AI model on the datasets comprises executing supervised learning operations on the datasets via the AI model.

19. The non-transitory computer readable storage media of claim 15, wherein training the AI model on the datasets comprises executing deep learning operations on the datasets via the AI model.

20. The non-transitory computer readable storage media of claim 15, wherein training the AI model on the datasets comprises training generic source models having strong generalizability and transferability to yield application-specific target models having superior task performance in the target task.

21. The non-transitory computer readable storage media of claim 15, wherein training the AI model on the datasets comprises training the AI model to generate as its output, one or more of:
a prediction of disease in a medical image;
a prediction of no disease in a medical image;
an image-level label not present in the source image;
an organ or lesion marker not present in the source image;
an organ or lesion bounding box not present in the source image; and
an organ or lesion mask not present in the source image.
